(19) United States
(12) Patent Application Publication
(10) Pub. No.: US 2023/0103637 A1
(43) Pub. Date: Apr. 6, 2023

(54) SEQUENCING OF VIRAL DNA FOR PREDICTING DISEASE RELAPSE

(71) Applicants: The Chinese University of Hong Kong, Shatin (HK); GRAIL, Inc., Menlo Park, CA (US)

(72) Inventors: Yuk-Ming Dennis Lo, Homantin (CN); Kwan Chee Chan, Mei Foo Sun Chuen (CN); Wai Kei Lam, Kowloon (CN); Chiu Tung Chan, Ma On Shan (CN)

(21) Appl. No.: 17/957,643

(22) Filed: Sep. 30, 2022

Related U.S. Application Data
(60) Provisional application No. 63/251,985, filed on Oct. 4, 2021.

(51) Int. Cl. C12Q 1/6886; C12Q 1/6858; C12Q 1/6869

(52) U.S. Cl. CPC C12Q 1/6886; C12Q 1/6858; C12Q 1/6869; C12Q 2600/118

(57) ABSTRACT

Various embodiments are directed to applications (e.g., classification of biological samples) of the analysis of the count and size of cell-free nucleic acids, e.g., plasma DNA and serum DNA, including nucleic acids from pathogens, such as viruses. Embodiments of one application can predict if a subject previously treated for a pathology will relapse at a future time point. Targeted sequencing (e.g., specifically designed capture probes, amplification primers) can be used to identify DNA across the entire viral genome.

### CROSS-REFERENCES TO RELATED APPLICATIONS

This application claims priority to U.S. Provisional Patent Application No. 63/251,985, entitled “Sequencing Of Viral DNA For Predicting Disease Relapse,” filed on Oct. 4, 2021, the contents of which are hereby incorporated by reference in their entirety for all purposes.

### BACKGROUND

The discovery that tumor cells release tumor-derived DNA into the blood stream has sparked the development of non-invasive methods capable of determining the presence, location and/or type of tumor in a subject using cell-free samples (e.g., plasma). Circulating cell-free DNA analysis has been shown to be of value in noninvasive monitoring of cancer treatment response and for the detection of cancer recurrence. However, conventional techniques can lack the sensitivity and/or specificity to detect disease relapse in subjects who previously completed a particular treatment. For example, real-time polymerase chain reaction (PCR) has been used to detect viral DNA in cell-free samples, and statistical values derived from the detected viral DNA were used to screen cancer patients. However, these conventional techniques suffer from low sensitivity in predicting disease relapse from post-treatment samples. Accordingly, there is a clinical need for methods having higher overall accuracy in predicting disease relapse in post-treatment samples.

### SUMMARY

Various embodiments are directed to applications (e.g., classification of biological samples) of the analysis of the count and/or size of cell-free nucleic acids, e.g., plasma DNA and serum DNA, including nucleic acids from pathogens, such as viruses. Embodiments of one application can predict if a subject previously treated for a pathology will relapse at a future time point. In some implementations, targeted sequencing (e.g., using capture probes or amplification primers) can be used to enrich for DNA of a viral genome (e.g., across all loci of viral genome or certain loci) relative to the genome of the subject (e.g., of a human genome). Targeted sequencing can be performed for the genome of the subject, e.g., so that only a portion of the human genome is analyzed, resulting in the amount of analyzed DNA in the subject being comparable to the amount of DNA analyzed in the subject, as opposed to being much greater. Such targeted sequencing can provide increased accuracy.

According to one embodiment, sequence reads obtained from a sequencing of the mixture of cell free nucleic acid molecules can be used to determine an amount of the sequence reads aligning to a viral reference genome corresponding to the virus. In one example, the amount of the sequence reads can represented by a proportion of sequence reads aligned to the viral reference genome relative to a total number of sequence reads. The amount of sequence reads aligning to the viral reference genome can be compared to a first cutoff value to predict relapse in post-treatment samples.

According to another embodiment, the count-based analysis can be combined with sizes of viral nucleic acid molecules (e.g., those aligning to a viral reference genome corresponding to the virus) for predicting relapse. A statistical value can represent a size ratio between: (1) a first proportion of sequence reads of nucleic acid molecules that align to the viral reference genome with the size within a given range; and (2) a second proportion of sequence reads of nucleic acid molecules that align to a human reference genome with the size within the given range. Disease relapse of the subject (e.g., a distant metastasis) can be determined by comparing the amount of sequence reads to the first cutoff value and the statistical value against a second cutoff value. In some instances, different first and second cutoff values are selected to increase the sensitivity and/or specificity for predicting disease relapse. In some instances, the statistical value corresponds to a size distribution of the plurality of nucleic acid molecules from the viral reference genome.

Other embodiments are directed to systems, portable consumer devices, and computer readable media associated with methods described herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts a schematic showing Epstein-Barr virus (EBV) DNA fragments from a nasopharyngeal cancer (NPC) cell being deposited into the bloodstream of a subject.

FIG. 2 depicts the concentration of plasma EBV DNA (copies/mL of plasma) in subjects with NPC and control subjects.

FIGS. 3A and 3B show plasma EBV DNA concentrations measured by real-time PCR for different groups of subjects.

FIG. 4 depicts the concentration of plasma EBV DNA (copies/mL of plasma) in subjects with early stage NPC and advanced stage NPC.

FIG. 5 shows plasma EBV DNA concentrations measured by real-time PCR for (left) subjects persistently positive for plasma EBV DNA but having no observable pathology, and (right) early-stage NPC patients identified by screening, as part of a validation analysis.

FIG. 6 shows plasma EBV DNA concentrations (copies/milliliter) measured by real-time PCR in subjects that are transiently positive or persistently positive for plasma EBV DNA (left or middle, respectively) but have no observable pathology, and subjects identified as having NPC.

FIG. 7 shows plasma EBV DNA concentrations (copies/milliliter) measured by real-time PCR in subjects that are transiently positive or persistently positive for plasma EBV DNA (left or middle, respectively) but have no observable pathology, and subjects identified as having NPC.

FIGS. 8A and 8B show the proportion of sequenced plasma DNA fragments mapped to the EBV genome for different groups of subjects.

FIG. 9 shows the proportion of reads mapped to the EBV genome in plasma for (left) subjects persistently positive for plasma EBV DNA but having no observable pathology, and (right) early-stage NPC patients identified by screening.

FIG. 10 depicts overall survival of subjects with various stages of NPC over time.

FIG. 11 shows plasma EBV DNA concentrations detected from post-treatment samples using real-time PCR.

FIG. 12 shows a graph that identifies overall survivals of NPC patients grouped according to plasma EBV DNA status determined by real-time PCR.

FIG. 13 shows a graph identifying a proportion of sequence reads detected from post-treatment samples using target-capture sequencing.

FIG. 14 is a flowchart illustrating a count-based method using sequence reads of viral nucleic acid fragments in cell-free mixture of a subject to predict disease relapse according to embodiments of the present invention.

FIG. 15 shows a graph identifying proportion and size ratio of EBV DNA detected from post-treatment samples using target-capture sequencing.

FIG. 16 is a flowchart for a method that combined a count-based and a size-based analysis of viral nucleic acid fragments to predict disease relapse according to embodiments of the present invention.

FIG. 17 shows receiver operating characteristic (ROC) curve for predicting disease relapse based on the analysis of plasma samples collected at 6 weeks after completion of curative-intent treatment for NPC patients.

FIG. 18 shows a graph identifying overall survival rates of NPC patients grouped according to the combined analysis using targeted-capture sequencing.

FIG. 19 shows a graph identifying overall survival rates of NPC patients grouped according to their estimated sequencing-based EBV levels.

FIG. 20 shows a graph identifying overall survival rates of NPC patients who had proportions of EBV DNA in plasma below 0.01%.

FIG. 21 shows plasma EBV DNA concentration by real-time PCR across NPC patients and pregnant women for which NPC was not identified.

FIG. 22 shows plasma EBV DNA concentration using count-based analysis across NPC patients and pregnant women for which NPC was not identified.

FIG. 23 shows count- and size-based analysis of plasma EBV DNA across NPC patients and pregnant women for which NPC was not identified.

FIG. 24 shows an example of a design of capture probes for targeted capture sequencing of subjects, according to embodiments of the present disclosure.

FIG. 25 shows a set of bar plots identifying plasma EBV DNA fractional concentration by sequencing of NPC samples via various target capture options.

FIG. 26 illustrates a system according to an embodiment of the present invention.

FIG. 27 shows a block diagram of an example computer system usable with system and methods according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (host vs. virus) or to healthy cells vs. tumor cells. The term “tissue” can generally refer to any group of cells found in the human body (e.g., heart tissue, lung tissue, kidney tissue, nasopharyngeal tissue, oropharyngeal tissue). In some aspects, the term “tissue” or “tissue type” may be used to refer to a tissue from which a cell-free nucleic acid originates. In one example, viral nucleic acid fragments may be derived from blood tissue, e.g., for Epstein-Barr Virus (EBV). In another example, viral nucleic acid fragments may be derived from tumor tissue, e.g., EBV or Human papillomavirus infection (HPV).

The term “sample”, “biological sample” or “patient sample” is meant to include any tissue or material derived from a living or dead subject. A biological sample may be a cell-free sample, which may include a mixture of nucleic acid molecules from the subject and potentially nucleic acid molecules from a pathogen, e.g., a virus. A biological sample generally comprises a nucleic acid (e.g., DNA or RNA) or a fragment thereof. The term “nucleic acid” may generally refer to deoxyribonucleic acid (DNA), ribonucleic acid (RNA) or any hybrid or fragment thereof. The nucleic acid in the sample may be a cell-free nucleic acid. A sample may be a liquid sample or a solid sample (e.g., a cell or tissue sample). The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. Stool samples can also be used. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed. The biological sample may be treated to physically disrupt tissue or cell structure (e.g., centrifugation and/or cell lysis), thus releasing intracellular components into a solution which may further contain enzymes, buffers, salts, detergents, and the like which are used to prepare the sample for analysis.

The terms “control”, “control sample”,“reference”,“reference sample”, “normal”, and “normal sample” may be interchangeably used to generally describe a sample that does not have a particular condition, or is otherwise healthy. In an example, a method as disclosed herein may be performed on a subject having a tumor, where the reference sample is a sample taken from a healthy tissue of the subject. In another example, the reference sample is a sample taken from a subject with the disease, e.g. cancer or a particular stage of cancer. A reference sample may be obtained from the subject, or from a database. The reference generally refers to a reference genome that is used to map sequence reads obtained from sequencing a sample from the subject.

The term “reference genome” generally refers to a haploid or diploid genome to which sequence reads from the biological sample and the constitutional sample can be aligned and compared. For a haploid genome, there is only one nucleotide at each locus. For a diploid genome, heterozygous loci can be identified, with such a locus having two alleles, where either allele can allow a match for alignment to the locus. A reference genome may correspond to a virus, e.g., by including one or more viral genomes.

The phrase “healthy,” as used herein, generally refers to a subject possessing good health. Such a subject demonstrates an absence of any malignant or non-malignant disease. A “healthy individual” may have other diseases or conditions, unrelated to the condition being assayed, that may normally not be considered “healthy”.

The terms “cancer” or “tumor” may be used interchangeably and generally refer to an abnormal mass of tissue wherein the growth of the mass surpasses and is not coordinated with the growth of normal tissue. A cancer or tumor may be defined as “benign” or “malignant” depending on the following characteristics: degree of cellular differentiation including morphology and functionality, rate of growth, local invasion, and metastasis. A “benign” tumor is generally well differentiated, has characteristically slower growth than a malignant tumor, and remains localized to the site of origin. In addition, a benign tumor does not have the capacity to infiltrate, invade, or metastasize to distant sites. A “malignant” tumor is generally poorly differentiated (anaplasia), has characteristically rapid growth accompanied by progressive infiltration, invasion, and destruction of the surrounding tissue. Furthermore, a malignant tumor has the capacity to metastasize to distant sites. “Stage” can be used to describe how advance a malignant tumor is. Early stage cancer or malignancy is associated with less tumor burden in the body, generally with less symptoms, with better prognosis, and with better treatment outcome than a late stage malignancy. Late or advanced stage cancer or malignancy is often associated with distant metastases and/or lymphatic spread.

A “level of pathology” can refer to the amount, degree, or severity of pathology associated with an organism, where the level can be as described above for cancer. Another example of pathology is a rejection of a transplanted organ. Other example pathologies can include autoimmune attack (e.g., lupus nephritis damaging the kidney or multiple sclerosis damaging the central nervous system), inflammatory diseases (e.g., hepatitis), fibrotic processes (e.g. cirrhosis), fatty infiltration (e.g. fatty liver diseases), degenerative processes (e.g. Alzheimer' s disease) and ischemic tissue damage (e.g., myocardial infarction or stroke). A heathy state of a subject can be considered a classification of no pathology. The pathology can be cancer.

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g. recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g. symptoms or other positive tests), has cancer.

The term “fragment” (e.g., a DNA fragment), as used herein, can refer to a portion of a polynucleotide or polypeptide sequence that comprises at least 3 consecutive nucleotides. A nucleic acid fragment can retain the biological activity and/or some characteristics of the parent polypeptide. A nucleic acid fragment can be double-stranded or single-stranded, methylated or unmethylated, intact or nicked, complexed or not complexed with other macromolecules, e.g. lipid particles, proteins. In an example, nasopharyngeal cancer cells can release fragments of Epstein-Barr Virus (EBV) DNA into the blood stream of a subject, e.g., a patient. These fragments can comprise one or more BamHI-W sequence fragments, which can be used to detect the level of tumor-derived DNA in the plasma. The BamHI-W sequence fragment corresponds to a sequence that can be recognized and/or digested using the Bam-HI restriction enzyme. The BamHI-W sequence can refer to the sequence 5′ -GGATCC-3′.

A tumor-derived nucleic acid can refer to any nucleic acid released from a tumor cell, including pathogen nucleic acids from pathogens in a tumor cell. For example, Epstein-Barr virus (EBV) DNA.

The term “assay” generally refers to a technique for determining a property of a nucleic acid. An assay (e.g., a first assay or a second assay) generally refers to a technique for determining the quantity of nucleic acids in a sample, genomic identity of nucleic acids in a sample, the copy number variation of nucleic acids in a sample, the methylation status of nucleic acids in a sample, the fragment size distribution of nucleic acids in a sample, the mutational status of nucleic acids in a sample, or the fragmentation pattern of nucleic acids in a sample. Any assay known to a person having ordinary skill in the art may be used to detect any of the properties of nucleic acids mentioned herein. Properties of nucleic acids include a sequence, quantity, genomic identity, copy number, a methylation state at one or more nucleotide positions, a size of the nucleic acid, a mutation in the nucleic acid at one or more nucleotide positions, and the pattern of fragmentation of a nucleic acid (e.g., the nucleotide position(s) at which a nucleic acid fragments). The term “assay” may be used interchangeably with the term “method”. An assay or method can have a particular sensitivity and/or specificity, and their relative usefulness as a diagnostic tool can be measured using ROC-AUC statistics.

The term “random sequencing,” as used herein, generally refers to sequencing whereby the nucleic acid fragments sequenced have not been specifically identified or predetermined before the sequencing procedure. Sequence-specific primers to target specific gene loci are not required.

In some embodiments, adapters are added to the end of a fragment, and the primers for sequencing attached to the adapters. Thus, any fragment can be sequenced with the same primer that attaches to a same universal adapter, and thus the sequencing can be random. Massively parallel sequencing may be performed using random sequencing.

A “sequence read” (or sequencing reads), as used herein, generally refers to a string of nucleotides sequenced from any part or all of a nucleic acid molecule. For example, a sequence read may be a short string of nucleotides (e.g., 20-150) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50x, 100x, etc., where “x” refers to the number of times a locus is covered with a sequence read. Sequencing depth can also be applied to multiple loci, or the whole genome, in which case x can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100x in sequencing depth.

The terms “size profile” and “size distribution” generally relate to the sizes of DNA fragments in a biological sample. A size profile may be a histogram that provides a distribution of an amount of DNA fragments at a variety of sizes. Various statistical parameters (also referred to as size parameters or just parameter) can distinguish one size profile to another. One parameter is the percentage of DNA fragment of a particular size or range of sizes relative to all DNA fragments or relative to DNA fragments of another size or range.

A “relative abundance” may generally refer to a ratio of a first amount of nucleic acid fragments having a particular characteristic (e.g., a specified length, ending at one or more specified coordinates/ending positions, or aligning to a particular region of the genome, aligning to a particular reference genome) to a second amount of nucleic acid fragments having a particular characteristic (e.g., a specified length, aligning to a particular region of the genome, aligning to a particular reference genome). In one example, relative abundance may refer to a ratio of (1) the number of DNA fragments that are from the viral reference genome having a size within a given range (e.g., 80-110 base pairs) and (2) the number of DNA fragments that are from a human reference genome with the size within the given range (e.g., 80-110 base pairs). In some aspects, “relative abundance” may correspond to a type of separation value that relates an amount (one value) of cell-free DNA molecules ending within one window of genomic positions to an amount (other value) of cell-free DNA molecules ending within another window of genomic positions. The two windows may overlap, but may be of different sizes. In other implementations, the two windows may not overlap. Further, the windows may be of a width of one nucleotide, and therefore be equivalent to one genomic position.

The term “classification” can refer to any number(s) or other characters(s) that are associated with a particular property of a sample. For example, a “+” symbol (or the word “positive”) could signify that a sample is classified as having deletions or amplifications. In another example, the term “classification” can refer to an amount of tumor tissue in the subject and/or sample, a size of the tumor in the subject and/or sample, a stage of the tumor in the subject, a tumor load in the subject and/or sample, presence of tumor metastasis in the subject, a relapse of a disease (e.g., the cancer) in the subject, and/or any other recurrence of symptoms of the disease after a period of improvement. For example, the classification can include remission, relapse, loco-regional failure, or distant metastasis of the disease. The classification can be binary (e.g., positive or negative) or have more levels of classification (e.g., a scale from 1 to 10 or 0 to 1).

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “true positive” (TP) can refer to subjects having a condition. True positive generally refers to subjects that have a tumor, a cancer, a pre-cancerous condition (e.g., a precancerous lesion), a localized or a metastasized cancer, or a non-malignant disease. True positive generally refers to subjects having a condition, and are identified as having the condition by an assay or method of the present disclosure.

The term “true negative” (TN) can refer to subjects that do not have a condition or do not have a detectable condition. True negative generally refers to subjects that do not have a disease or a detectable disease, such as a tumor, a cancer, a pre-cancerous condition (e.g., a precancerous lesion), a localized or a metastasized cancer, a non-malignant disease, or subjects that are otherwise healthy. True negative generally refers to subjects that do not have a condition or do not have a detectable condition, or are identified as not having the condition by an assay or method of the present disclosure.

The term “false positive” (FP) can refer to subjects not having a condition. False positive generally refers to subjects not having a tumor, a cancer, a pre-cancerous condition (e.g., a precancerous lesion), a localized or a metastasized cancer, a non-malignant disease, or are otherwise healthy. The term false positive generally refers to subjects not having a condition, but are identified as having the condition by an assay or method of the present disclosure.

The term “false negative” (FN) can refer to subjects that have a condition. False negative generally refers to subjects that have a tumor, a cancer, a pre-cancerous condition (e.g., a precancerous lesion), a localized or a metastasized cancer, or a non-malignant disease. The term false negative generally refers to subjects that have a condition, but are identified as not having the condition by an assay or method of the present disclosure.

The terms “sensitivity” or “true positive rate” (TPR) can refer to the number of true positives divided by the sum of the number of true positives and false negatives. Sensitivity may characterize the ability of an assay or method to correctly identify a proportion of the population that truly has a condition. For example, sensitivity may characterize the ability of a method to correctly identify the number of subjects within a population having cancer. In another example, sensitivity may characterize the ability of a method to correctly identify one or more markers indicative of cancer.

The terms “specificity” or “true negative rate” (TNR) can refer to the number of true negatives divided by the sum of the number of true negatives and false positives. Specificity may characterize the ability of an assay or method to correctly identify a proportion of the population that truly does not have a condition. For example, specificity may characterize the ability of a method to correctly identify the number of subjects within a population not having cancer. In another example, specificity may characterize the ability of a method to correctly identify one or more markers indicative of cancer.

The term “ROC” or “ROC curve” can refer to the receiver operator characteristic curve. The ROC curve can be a graphical representation of the performance of a binary classifier system. For any given method, an ROC curve may be generated by plotting the sensitivity against the specificity at various threshold settings. The sensitivity and specificity of a method for detecting the presence of a tumor in a subject may be determined at various concentrations of tumor-derived nucleic acid in the plasma sample of the subject. Furthermore, provided at least one of the three parameters (e.g., sensitivity, specificity, and the threshold setting), and ROC curve may determine the value or expected value for any unknown parameter. The unknown parameter may be determined using a curve fitted to the ROC curve. The term “AUC” or “ROC-AUC” generally refers to the area under a receiver operator characteristic curve. This metric can provide a measure of diagnostic utility of a method, taking into account both the sensitivity and specificity of the method. Generally, ROC-AUC ranges from 0.5 to 1.0, where a value closer to 0.5 indicates the method has limited diagnostic utility (e.g., lower sensitivity and/or specificity) and a value closer to 1.0 indicates the method has greater diagnostic utility (e.g., higher sensitivity and/or specificity). See, e.g., Pepe et al, “Limitations of the Odds Ratio in Gauging the Performance of a Diagnostic, Prognostic, or Screening Marker,” Am. J. Epidemiol 2004, 159 (9): 882-890, which is entirely incorporated herein by reference. Additional approaches for characterizing diagnostic utility using likelihood functions, odds ratios, information theory, predictive values, calibration (including goodness-of-fit), and reclassification measurements are summarized according to Cook, “Use and Misuse of the Receiver Operating Characteristic Curve in Risk Prediction,” Circulation 2007, 115: 928-935, which is entirely incorporated herein by reference.

“Negative predictive value” or “NPV” may be calculated by TN/(TN+FN) or the true negative fraction of all negative test results. Negative predictive value is inherently impacted by the prevalence of a condition in a population and pre-test probability of the population intended to be tested. “Positive predictive value” or “PPV” may be calculated by TP/(TP+FP) or the true positive fraction of all positive test results. It is inherently impacted by the prevalence of the disease and pre-test probability of the population intended to be tested. See, e.g., O'Marcaigh A S, Jacobson R M, “Estimating The Predictive Value Of A Diagnostic Test, How To Prevent Misleading Or Confusing Results,” Clin. Ped. 1993, 32(8): 485-491, which is entirely incorporated herein by reference.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art.

Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited.

The specific examples described herein are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

### DETAILED DESCRIPTION

Viral infection can be an etiological factor for various types of cancers. For example, Epstein-Barr virus (EBV) has been known to cause nasopharyngeal carcinoma (NPC), certain types of lymphoma, and gastric cancer. Thus, the analysis of plasma EBV DNA by real-time PCR has been shown to be useful for the detection of NPC (Lo et al. Cancer Res. 1999; 59:1188-91). Furthermore, the detection of EBV DNA in the plasma of NPC patients by real-time PCR after curative-intent treatment has been shown to be strongly associated with disease recurrence (Chan et al. J Natl Cancer Inst. 2002; 94:1614-9). In view of this predictive value, a randomized control trial has been carried out to investigate if adjuvant chemotherapy would be useful for improving the outcome of NPC patients who have detectable EBV DNA after curative-intent therapy (Chan et al. J Clin Oncol. 2018; 36: 3091-3100). Specifically, NPC patients who had detectable plasma EBV DNA after curative-intent treatment were randomized to either receive adjuvant chemotherapy or clinical observation (i.e. the standard of care), in which the plasma EBV DNA was detected using real-time EBV DNA.

The results, however, indicated that these two groups did not show any significant difference in the relapse-free survival. These results could have been due to low sensitivity of real-time PCR in identifying the NPC patients who had detectable plasma EBV DNA. For example, the post-treatment plasma EBV DNA analysis by real-time PCR only identified 48% of the cases that would subsequently relapse. The sensitivities for predicting locoregional failure and distant metastasis were 42% and 53%, respectively. Therefore, although it has been shown the presence of detectable EBV DNA in plasma was associated with disease relapse, real-time PCR was found to be an ineffective method for detecting EBV DNA in post-treatment samples for prediction of relapse.

To address at least the above deficiencies, the present techniques can use sequencing to accurately detect viral DNA in post-treatment samples for detecting relapse of a disease, such as cancer. In particular, the present techniques are directed to an improved method of predicting relapse of the disease by detecting low amounts of viral DNA that would have been incapable of being detected via other techniques. For example, real-time PCR may produce results in which detected viral DNA levels are expected to be close to zero, but the present techniques can use such information to accurately predict relapse of cancer. Further, the present techniques can analyze sequence reads corresponding to the entire viral genome to predict relapse, rather than focusing on sequence reads that align to specific regions of the viral genome. By accurately predicting disease relapse of subjects, the present techniques can facilitate early intervention and selection of appropriate treatments to improve disease outcome and overall survival rates of subjects. For example, an intensified chemotherapy can be selected for subjects, in the event their corresponding samples are predictive of disease relapse.

Below, the first section describes techniques that can be used to diagnose a subject via detection of pathogen DNA, i.e., determine whether a subject currently has the pathology, as opposed to determining a prognosis of a relapse. Later sections describe techniques and improved techniques for determining a classification of a relapse for a subject.

### I. VIRAL DNA IN CELL-FREE SAMPLES

Pathogens can invade a cell. For example, viruses such as EBV can exist within cells. These pathogens can release their nucleic acids (e.g., DNA or RNA). The nucleic acids are often released from cells in which the pathogen has caused some pathology, e.g., cancer.

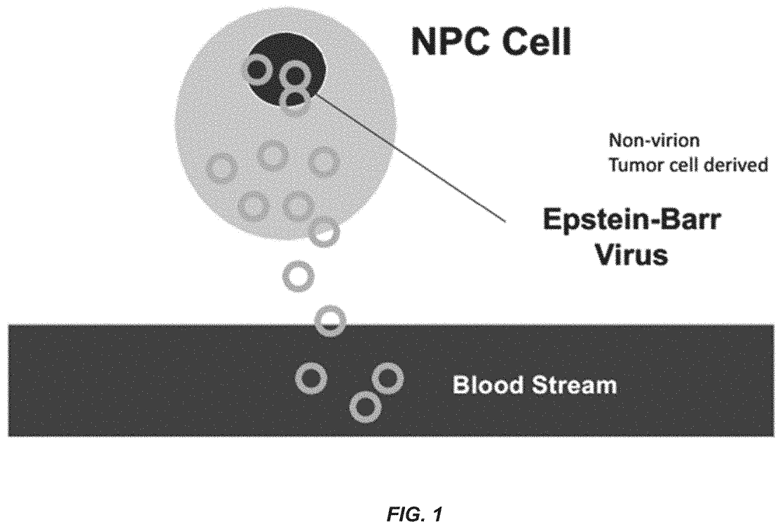

FIG. 1 shows an NPC cell that includes EBV. An NPC cell may include many copies of the virus, e.g., 50. FIG. 1 shows nucleic acid fragments 110 of the EBV genome being released (e.g., when the cell dies) into the blood stream. Although nucleic acid fragments 110 are depicted as circular (e.g., as the EBV genome is circular), the fragments would just be part of the EBV genome. Thus, an NPC cell can deposit fragments of the EBV DNA into the bloodstream of a subject. This tumor marker can be useful for the monitoring (Lo et al. Cancer Res 1999; 59: 5452-5455) and prognostication (Lo et al. Cancer Res 2000; 60: 6878-6881) of NPC.

### A. Relation of Certain Viruses to Various Cancers

Viral infections are implicated in a number of pathological conditions. For example, EBV infection is closely associated with NPC and natural killer (NK) T-cell lymphoma, Hodgkin lymphoma, gastric cancer, and infectious mononucleosis. Hepatitis B virus (HBV) infection and hepatitis C virus (HCV) infection are associated with increased risks of developing hepatocellular carcinoma (HCC). Human papillomavirus infection (HPV) are associated with increased risks of developing cervical cancer (CC) and head and neck squamous cell carcinoma (HNSCC).

However, not all subjects that have such an infection will get an associated cancer. The source of the plasma EBV DNA must be different in persons without NPC. Unlike the persistent release of EBV DNA into the circulation from NPC cells, the source of EBV DNA only contributes such DNA transiently in the persons without NPC.

### B. Detecting Viral DNA in Cell-Free Samples

As examples, the nucleic acids of the pathogen found in the sample (e.g., plasma or serum) may be: (1) released from tumor tissues; (2) released from a non-cancer cell, e.g. rest B cells carrying EBV; and (3) contained in a virion.

The pathogenesis of NPC is closely associated with EBV infection. In endemic areas of NPC, e.g. South China, almost all NPC tumor tissues harbor EBV genomes. In this regard, plasma EBV DNA has been established as a biomarker for NPC (Lo et al. Cancer Res 1999; 59:1188-91). It has been shown that plasma EBV DNA is useful for detecting residual disease in NPC subjects after curative-intent treatment (Lo et al. Cancer Res 1999; 59:5452-5 and Chan et al. J Natl Cancer Inst 2002; 94:1614-9). The plasma EBV DNA in NPC subjects has been shown to be short DNA fragments of less than 200 bp and is thus unlikely to have derived from intact virion particles (Chan et al. Cancer Res 2003, 63:2028-32).

### 1. qPCR Assay for Late Stage

A real-time quantitative PCR assay (qPCR) can detect late stage NPC using specific regions of the EBV genome, specifically two regions of the EBV genome, the BamHI-W and the EBNA-1 regions. There can be about six to twelve repeats of the BamHI-W fragments in each EBV genome and there can be approximately 50 EBV genomes in each NPC tumor cell (Longnecker et al. Fields Virology, 5th Edition, Chapter 61 “Epstein-Barr virus”; Tierney et al. J Virol. 2011; 85: 12362-12375). In other words, there can be on the order of 300-600 (e.g., about 500) copies of the PCR target in each NPC tumor cell.

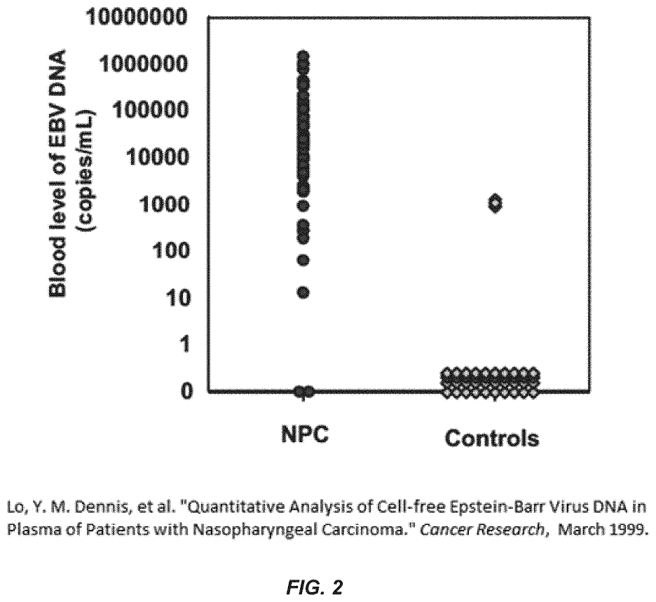

FIG. 2 shows a comparison of plasma cell-free EBV DNA in NPC and control subjects. The categories (NPC and control subjects) are plotted on the X axis. The Y axis denotes the concentration of cell-free EBV DNA (copies of EBV DNA/ml of plasma) detected by the BamHI-W region PCR system. Similar results were obtained using the EBNA-1 PCR that showed a strong correlation with the BamHI-W region PCR data (Spearman rank order correlation, correlation coefficient 5 0.918; P, 0.0005).

As shown in FIG. 2, cell-free EBV DNA was detectable in the plasma of 96% (55 of 57) of nasopharyngeal carcinoma (NPC) patients (median concentration, 21058 copies/ml) and 7% (3 of 43) of controls (median concentration, 0 copies/ml).

In a further analysis, Table 1 shows the number of different types of samples analyzed. In the initial analysis (cohort 1), six subjects presenting with symptoms compatible with NPC, including neck lumps, hearing loss and epistaxis were recruited from the ear-nose and throat (ENT) clinic. The NPC subjects in cohort 1 have advanced disease (late stage) than those examined in other cohorts that did not present with symptoms. Historical data from the Hong Kong Cancer Registry shows that 80% of individuals presenting with symptoms and later confirmed to have NPC had advanced stage NPC at the time of presentation for medical care.

TABLE 1

| | Number of |
|---|---|
| Type of samples | samples |
| Non-NPC subjects with detectable plasma EBV DNA at | 5 |
| enrollment to the study but undetectable plasma EBV DNA | |
| approximately four weeks later. For these subjects, the | |
| samples collected at enrollment were analyzed. These | |
| subjects are denoted as “transiently positive”. | |
| Non-NPC subjects with persistently detectable plasma EBV | 9 |
| DNA at enrollment and approximately four weeks later. For | |
| these subjects, the samples collected at enrollment were | |
| analyzed. These subjects are denoted as “persistently | |
| positive”. | |
| NPC subjects | 6 |
| EBV-positive lymphoma subjects (two with NK T-cell | 3 |
| lymphoma and one with Hodgkin lymphoma) | |
| Subject with infectious mononucleosis | 1 |

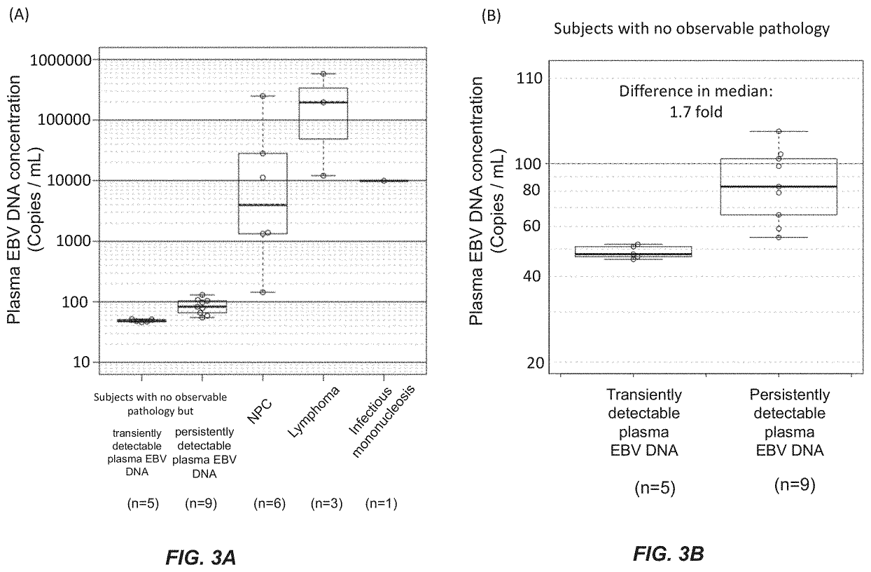

FIGS. 3A and 3B show plasma EBV DNA concentrations measured by real-time PCR for different groups of subjects. As shown in FIG. 3A, plasma EBV DNA concentrations were higher in subjects with NPC, lymphoma, and infectious mononucleosis compared with those with detectable plasma EBV DNA, but without any observable pathology. As shown in FIG. 3B, for those subjects with detectable plasma EBV DNA at enrollment but without any observable pathology, the plasma EBV DNA concentration measured at enrollment was higher in the subjects with persistently positive results compared with those who would become negative in the follow-up test (i.e. with transiently detectable plasma EBV DNA) (p=0.002, Mann-Whitney test).

### 2. qPCR Results for Early Stage

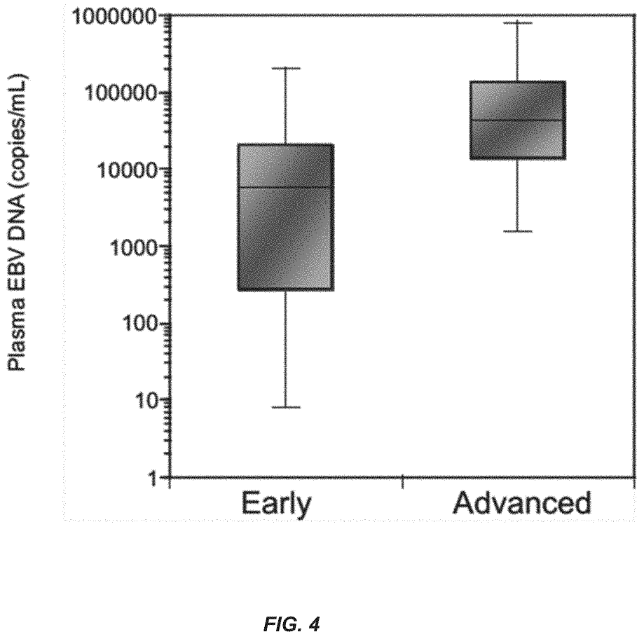

FIG. 4 depicts the concentration of plasma EBV DNA (copies/mL of plasma) in subjects with early stage NPC and advanced stage NPC. As shown in FIG. 4, this test plasma cell-free EBV DNA levels in advanced NPC cases (median, 47,047 copies/ml; interquartile range, 17,314-133,766 copies/ml) were significantly higher than those in early-stage NPC cases (median, 5,918 copies/ml; interquartile range, 279-20,452 copies/ml; Mann-Whitney rank-sum test, P<0.001).

As mentioned herein, the detection of late stage NPC is not as useful as an early stage detection. The utility of a plasma EBV DNA analysis using real-time PCR for BamHI-W fragments was investigated for the detection of early NPC in asymptomatic subjects. (Chan et al. Cancer 2013; 119:1838-1844). In a population study with 1,318 participants, plasma EBV DNA levels were measured to investigate whether EBV DNA copy number can be useful for NPC surveillance. 69 participants (5.2%) had detectable levels of plasma EBV DNA, of 3 participants ultimately were clinically diagnosed, using nasal endoscopy and magnetic resonance imaging, as having NPC. Thus, the positive predictive value (PPV) of a single plasma EBV DNA test in this study is about 4%, calculated as the number of patients truly having NPC (n=3) divided by the sum of number of patients truly having NPC and the number of patients falsely identified as having NPC (n=66).

A larger study of 20,174 asymptomatic Chinese males aged between 40 to 62 years was performed. Out of the recruited 20,174, there were 1,112 subjects (5.5%) who had detectable plasma EBV DNA from baseline PCR tests. Among them, 34 subjects were later confirmed to have NPC. For the remaining 1,078 non-cancer subjects, 803 subjects had ‘transiently positive’ plasma EBV DNA results (i.e., positive at baseline but negative at follow-up) and 275 had ‘persistently positive’ plasma EBV DNA results (i.e., positive at both baseline and follow-up). A validation analysis was first performed with a subset of the data.

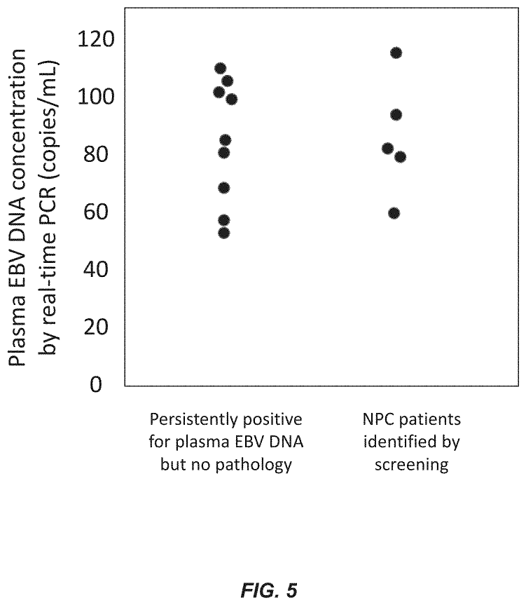

FIG. 5 shows plasma EBV DNA concentrations measured by real-time PCR for (left) subjects persistently positive for plasma EBV DNA but having no observable pathology, and (right) early-stage NPC patients identified by screening, as part of a validation analysis. Five of the 34 NPC subjects who were identified through the screening of 20,174 asymptomatic subjects were included in this validation analysis. These 5 subjects were asymptomatic when they joined the study. The plasma samples of these 5 subjects in cohort 2 were persistently positive for EBV DNA, and NPC was subsequently confirmed by endoscopy and Mitt These 5 asymptomatic NPC cases were of early stage unlike the 6 NPC subjects in cohort 1 who presented to ENT clinics with symptoms and were diagnosed with advanced stage NPC.

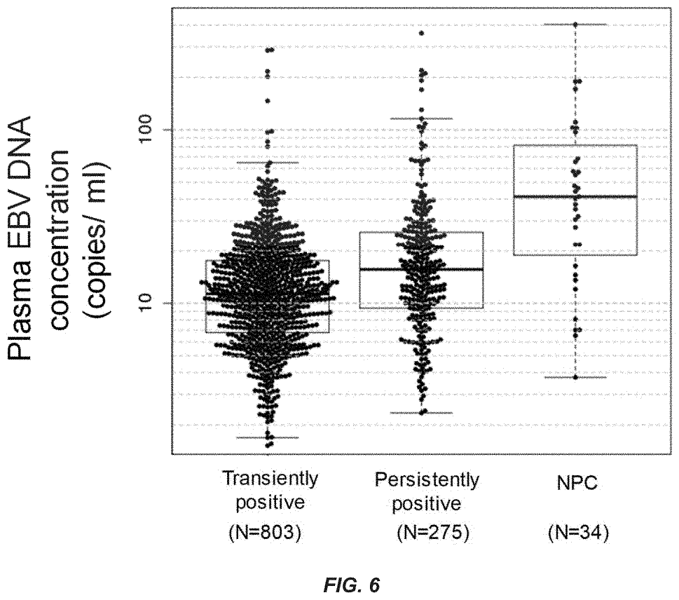

FIG. 6 shows a box and whiskers plot of the concentration of EBV DNA fragments (copies per milliliter) from the plasma of subjects that are transiently positive (n=803) or persistently positive (n=275) for plasma EBV DNA (left or middle, respectively) but have no observable pathology, and subjects identified as having NPC (n=34). FIG. 6 shows results for all of the subjects of the 1,112 subjects who had detectable plasma EBV DNA from baseline PCR tests. The concentration of EBV DNA fragments (copies per milliliter) was measured by real-time PCR analysis.

Plasma EBV DNA results were expressed as ‘positive’ or ‘negative’. The quantitative levels of the plasma EBV DNA concentrations between the groups are measured by real-time PCR (FIG. 6). The mean plasma EBV DNA concentration of the NPC group (942 copies/mL; interquartile range (IQR), 18 to 68 copies/mL) was significantly higher than those of the ‘transiently positive’ group (16 copies/mL; IQR, 7 to 18 copies/mL) and ‘persistently positive’ group (30 copies/mL; IQR, 9 to 26 copies/mL) (P<0.0001, Kruskal-Wallis test). However, there is much overlap in the plasma EBV DNA concentrations among the three groups (FIG. 6).

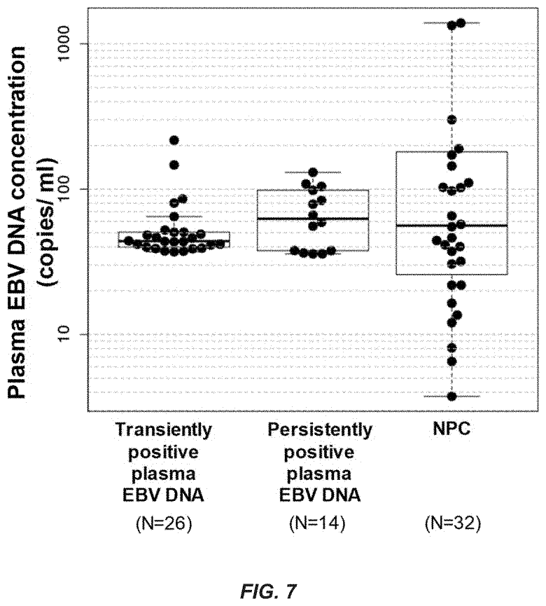

FIG. 7 shows plasma EBV DNA concentrations (copies/milliliter) measured by real-time PCR in subjects that are transiently positive or persistently positive for plasma EBV DNA (left or middle, respectively) but have no observable pathology, and subjects identified as having NPC. In this cohort of 72 subjects, there was no statistically significant difference in the plasma EBV DNA concentrations measured by real-time PCR among different groups of subjects (p-value=0.19; Kruskal-Wallis test).

### 3. Two Assay Analysis for Early Stage

The real-time polymerase chain reaction (PCR) assay used in the prospective screening study was shown to be highly sensitive to the detection of plasma EBV DNA even from small tumors. However, the test specificity suffers. The peak age-specific incidence of NPC in Hong Kong is 40 per 100,000 persons but approximately 5% of healthy population has detectable levels of EBV DNA in plasma. The screening study yielded a positive predictive value (PPV) of 3.1% when plasma EBV DNA assessment by the real-time PCR assay was performed on one occasion per participant.

Given the low PPV of the real-time PCR assay, the utility of two assays at two times was investigated. For example, the above previous study showed that EBV DNA tended to be persistently detectable in the plasma of NPC subjects, but appeared transiently in the plasma of non-cancer subjects.

A study screened 20,174 subjects without symptoms of NPC using plasma EBV DNA analysis. Subjects with detectable plasma EBV DNA were retested in approximately 4 weeks later with a follow-up plasma EBV DNA analysis. Subjects with persistently positive results on the two serial analyses were further investigated with nasal endoscopic examination and magnetic resonance imaging (MM) of the nasopharynx. Out of the 20,174 subjects recruited, 1,112 were positive for plasma EBV DNA at enrollment. Among them, 309 were persistently positive on the follow-up test. Within the cohort of subjects who were persistently positive for EBV DNA in plasma, 34 were subsequently confirmed of having NPC after being investigated with nasal endoscopic examination and Mill.

The two time-point testing approach indeed reduced the false-positive rate from 5.4% to 1.4% with a resultant PPV of 11.0%. These results showed that the retesting of the subjects with initial positive plasma EBV DNA results could differentiate NPC subjects from those with transiently positive results and substantially reduce the proportion of subjects requiring the more invasive and costly investigations, namely endoscopy and Mill. However, the sequential testing of plasma EBV DNA requires the collection of an additional blood sample from subjects with initial positive results, which can present logistical challenges.

### C. Analysis of Early Stage and Late Stage Cancers Using Viral DNA

In some cases, an assay to screen for a condition (e.g., tumor, e.g., NPC) after an initial assay (e.g., qPCR assay) or instead of qPCR can comprise using massively parallel sequencing to assess proportion of sequence reads from a sample that map to a viral reference genome (e.g., EBV).

To analyze the cell-free viral DNA in plasma, targeted sequencing (e.g., specifically designed capture probes, amplification primers) can be used. These capture probes covered the whole EBV genome, the whole HBV genome, the whole HPV genome and multiple genomic regions in the human genome (including regions on chr1, chr2, chr3, chr5, chr8, chr15 and chr22). For each plasma sample analyzed, DNA was extracted from 4 mL plasma using the QIAamp DSP DNA blood mini kit. For each case, all extracted DNA was used for the preparation of the sequencing library using the KAPA library preparation kit. Twelve cycles of PCR amplification were performed on the sequencing library using the KAPA PCR amplification kit. The amplification products were captured using the SEQCAP-EZ kit (Nimblegen) using the custom-designed probes covering the viral and human genomic regions stated above. After target capturing, 14 cycles of PCR amplification were performed and the products were sequenced using the Illumina NextSeq platform. For each sequencing run, four to six samples with unique sample barcodes were sequenced using the paired-end mode. Each DNA fragments would be sequenced 75 nucleotides from each of the two ends. After sequencing, the sequenced reads would be mapped to an artificially combined reference sequence which consists of the whole human genome (hg19), the whole EBV genome, the whole HBV genome and the whole HPV genome. Sequenced reads mapping to unique position in the combined genomic sequence would be used for downstream analysis. The median number of uniquely mapped reads is 53 million (range: 15˜141 million).

### 1. Late Stage

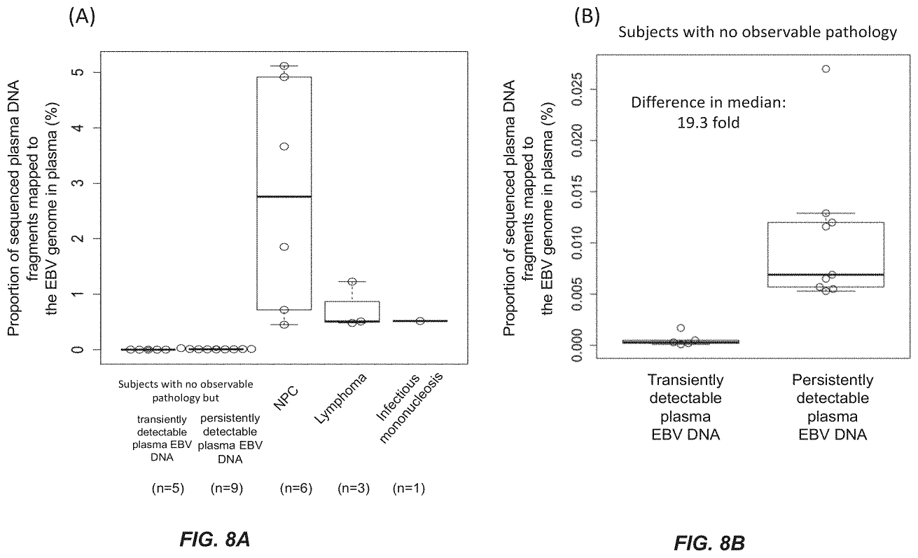

FIGS. 8A and 8B show the proportion of sequenced plasma DNA fragments mapped to the EBV genome in plasma for different groups of subjects. The subjects correspond to cohort 1, same as FIGS. 3A and 3B.

As shown in FIG. 8A, using massively parallel sequencing following target capture, the proportions of reads uniquely mapped to the EBV genome were higher in subjects with NPC, lymphoma and infectious mononucleosis compared with those with detectable plasma EBV DNA at enrollment but without any observable pathology. As shown in panel B, for those subjects with detectable plasma EBV DNA at enrollment but without any observable pathology, the proportion of reads mapped to the EBV genome measured at enrollment was higher in the subjects with persistently positive results compared with those who would become negative in the follow-up test (i.e. with transiently detectable plasma EBV DNA) (p=0.002, Mann-Whitney test). The difference between subjects with transiently and persistently positive results is greater using the measurement of the proportion of reads uniquely mapped to the EBV genome compared with the concentration of plasma EBV DNA measured using real-time PCR (19.3 folds vs 1.7 folds).

Elevated plasma EBV DNA is associated with NPC. Previous studies compared NPC cases and healthy controls who are mostly negative for plasma EBV DNA. FIGS. 3A, 3B, 8A, and 8B provide a quantitative comparison between NPC cases and the non-NPC cases who are false-positive for plasma EBV DNA. Techniques described below allow for increased accuracy in discriminating between subjects with a pathology and those without, thereby reducing false-positives. In the context of EBV DNA, the term “false-positive” can mean that the subject has detectable plasma EBV DNA but the subject does not have NPC (an example of a pathology associated with the pathogen). The presence of plasma EBV DNA is true, but the identification of the associated pathology (e.g., NPC) may be false.

### 2. Early Stage

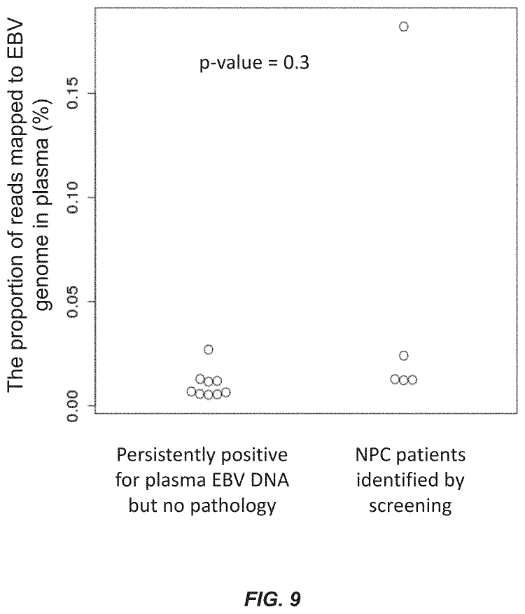

FIG. 9 shows the proportion of reads mapped to the EBV genome in plasma for (left) subjects persistently positive for plasma EBV DNA but having no observable pathology, and (right) early stage NPC subjects. The subjects correspond to cohort 2, same as FIG. 5.

The plasma samples were sequenced after target enrichment as described above. For the five NPC subjects in cohort 2, while their plasma samples were persistently positive for EBV DNA, the EBV DNA concentration did not show significant difference compared with the 9 subjects with false-positive plasma EBV DNA results based on real-time PCR analysis (P=0.7, Mann-Whitney test). Plasma EBV DNA concentration is known to correlate with the stage of NPC. Thus, it is not unexpected that the early stage NPC subjects had lower levels of plasma EBV DNA.

The proportion of sequenced plasma DNA reads mapped to the EBV genome were not significantly different between the false-positive cases and the cohort 2 NPC cases.

These initial data suggest that the approaches shown in FIGS. 5 and 9 may not work well in differentiating false-positives from the early stage NPCs for identifying a relapse.

### D. Benefits of Early Stage Diagnosis

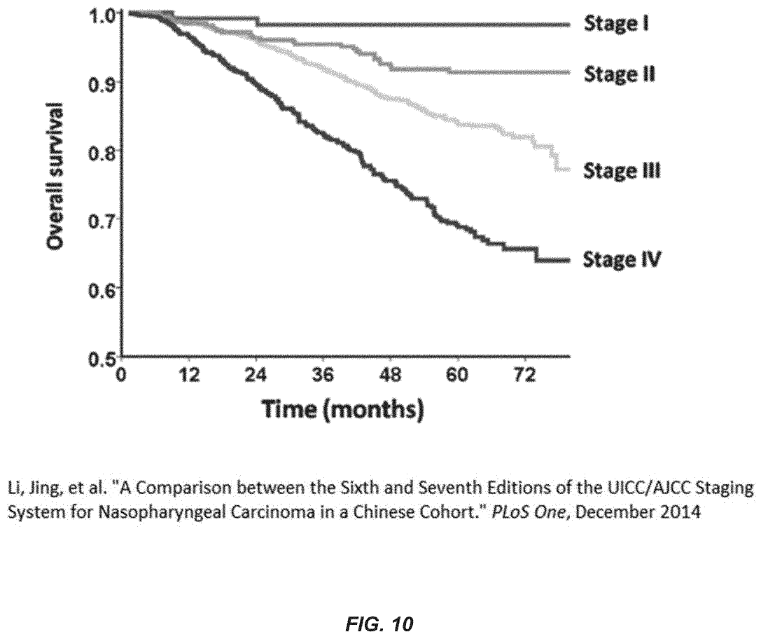

FIG. 10 depicts the overall survival of NPC patients at various stages of cancer, and the stage distribution of NPC in Hong Kong, respectively. Such a benefit of early diagnosis can also apply to a detection of relapse, as a subject can be monitored more acutely (e.g., endoscopy, PET-CT scan) to determine when relapse has actually occurred, and thus when treatment should be applied. If there is a higher likelihood of relapse, a subject could be tested more frequently. Accordingly, some embodiments can be useful in reducing the number of patients that reach a higher stage of cancer, thereby increasing their overall survival probability.

### II. REAL-TIME PCR FOR PREDICTING DISEASE RELAPSE

### A. Sensitivity of Real-Time PCR for Predicting Relapse

As noted above, real-time PCR can be used to detect and differentiate different conditions associated with viral infections by analyzing the levels of circulating viral DNA. For example, real-time PCR techniques have been attempted in post-treatment samples for predicting relapse of a pathology (e.g., cancer) of a subject. These attempts were performed based on the fact that, if viral DNA is identified in the subject after a treatment has been completed, some subjects develop disease relapse in few years.

However, real-time PCR does not predict the presence of residual viral DNA in post-treatment samples at desirable sensitivity levels. For example, real-time PCR was able to detect around 40 to 50% of the subjects who actually relapsed after completing the treatment. Performance of the real-time PCR further deteriorates when detecting for specific classifications of relapse in post-treatment samples. For example, real-time PCR has lower sensitivity levels (of 33.3%) when it is used to predict local relapse, compared to detecting distant relapse in the post-treatment samples.

One of the contributing factors of low sensitivity levels of real-time PCR can be attributed to its inability to detect low amounts of circulating viral DNA in the subject. If a subject has cancer, viral DNA would be released into the subject's circulation. For subjects who have completed a particular treatment but are still expected to relapse, one would initially expect a very low concentration of viral DNA as the residual tumor could be small as a result of the treatment. Despite this characteristic, real-time PCR is configured such that it detects only a specific region or a few specific regions of the viral genome (e.g., around 70-100 base pairs). When the residual tumor is small and the viral DNA concentration is very low, it is possible that real-time PCR would give a false-negative result as the specific region(s) of the viral genome the real-time PCR targets is not present in the sample or is present at a concentration below the detection limit of the assay.

For predicting post-treatment relapse, capturing the entire viral genome can be beneficial to detect low amounts of residual viral DNA. Embodiments of the present disclosure recognize that using probes allows the capture of all of the viral sequences (e.g., about 170 kilobases) in the sample. Once the entire viral genome is captured, sequencing can be performed to identify various characteristics of the nucleic acid molecules to detect relapse of nasopharyngeal carcinoma (NPC), certain types of lymphoma and gastric cancer at higher sensitivity levels.

### B. Example

Post-treatment samples from 737 patients treated for nasopharyngeal carcinoma were collected. (Chan et al. J Clin Oncol. 2018; 36: 3091-3100). Each of the post-treatment samples initially had a histologic diagnosis of locoregionally advanced NPC of Union for International

Cancer Control (UICC; 6th edition) stage IIB, III, IVA, or IVB, but did not have clinical evidence of persistent locoregional disease or distant metastasis after completion of primary radiation therapy or chemoradiation therapy. In addition, post-treatment venous samples were collected at 6 to 8 weeks after the completion of treatment. The median follow-up interval was 6.6 years. Among the 737 patients, 643 patients (87%) had continuous clinical remission during the first-year post treatment, while 94 patients (13%) reported relapse of the disease. Among the 94 relapsed patients, 24 patients (26%) experienced locoregional failure and 70 patients (74%) experienced distant metastasis.

Real-time PCR targeting the EBV Bam-HI W fragment was performed to identify viral DNA concentrations (copies/milliliter) for each post-treatment sample. Then, the viral DNA concentration of each post-treatment sample was plotted along its corresponding relapse classification. The classifications included a first classification corresponding to patients who had continuous clinical remission and a second classification corresponding to patients who relapsed within 1-year after completing treatment. Under the relapse classification, the patients were further divided into the following: (1) a local recurrence (LR) classification which corresponds a local relapse; and (2) a distant metastasis (DM) classification which indicates cancer having metastasized to other organs.

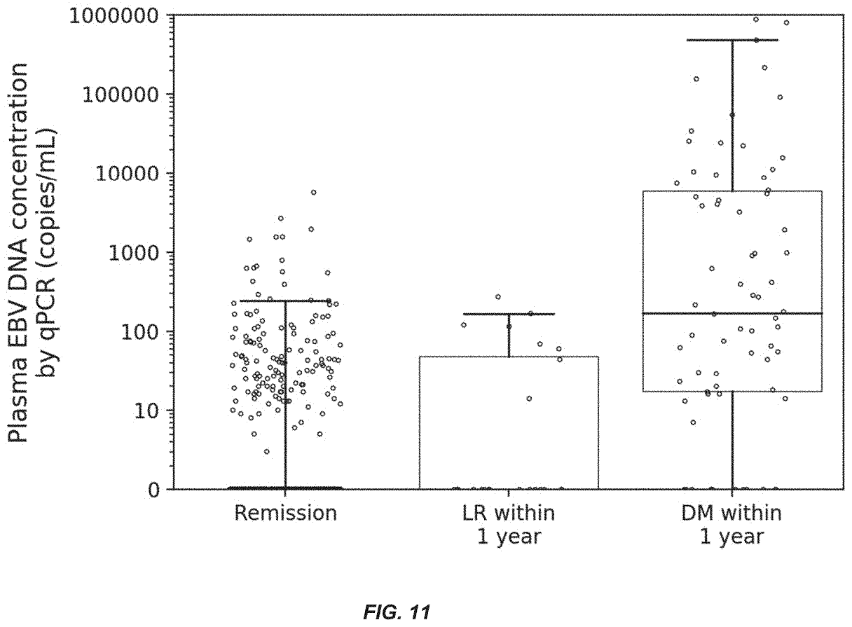

FIG. 11 shows plasma EBV DNA concentrations detected from post-treatment samples using real-time PCR. The x-axis represents a classification associated with a post-treatment sample, and the y-axis represents viral DNA concentration for the corresponding post-treatment sample. In 643 post-treatment samples corresponding to patients under remission classification, plasma EBV DNA was detected in 137 (21%) of the samples. In 94 post-treatment samples under the relapse classification, 66 (70%) had detectable plasma EBV DNA. The 94 relapsed samples were then further divided into LR and DM classifications. For 70 post-treatment samples under the DM classification, 58 (83%) had detectable plasma EBV DNA. For 24 post-treatment samples under the LR classification, only 8 (33%) had detectable plasma EBV DNA.

Thus, the real-time PCR was only able to detect viral DNA in 70% of patients who experienced relapse of the disease. Such low sensitivity thus demonstrates that real-time PCR may not be the most effective technique for detecting viral DNA in post-treatment samples.

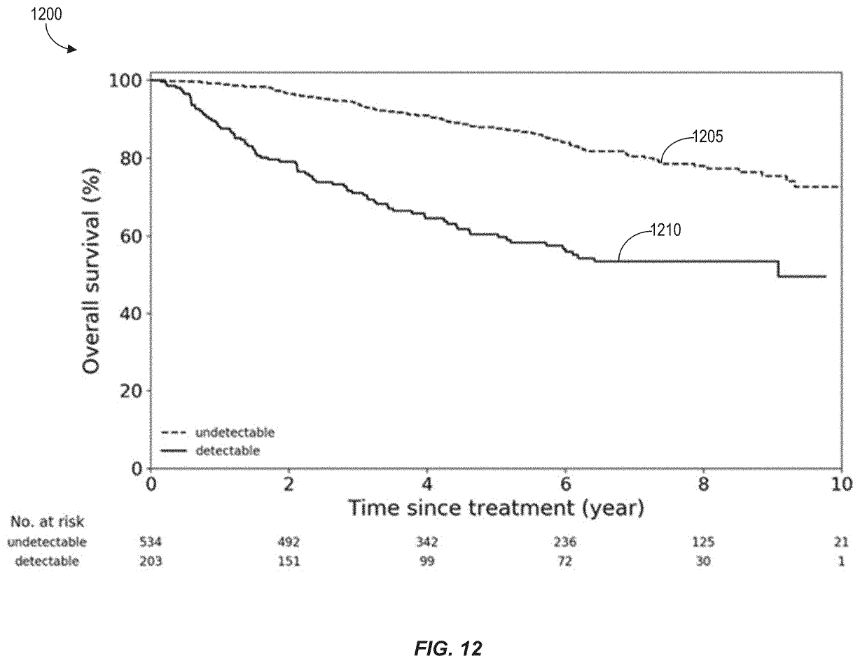

FIG. 12 shows a graph 1200 that identifies overall survivals of NPC patients grouped according to plasma EBV DNA status determined by real-time PCR. Kaplan-Meier survival analysis was used to analyze the survivals of different patient groups. Patients with EBV DNA under a predefined threshold in plasma were denoted as “undetectable”, while patients with plasma EBV DNA above the predefined threshold. were identified as “detectable”. In this example, the predefined threshold was set to 20 copies/mL. The overall survival rate for patients with undetectable plasma EBV DNA is represented by a dashed line 1205, and the overall survival rate for patients with detectable plasma EBV is represented by a solid line 1210. The overall survival of patients with detectable plasma EBV DNA by real-time PCR was significantly poorer than those without detectable plasma EBV DNA (p<0.0001, log-rank test) (fig. a). The 5-year survival rate of patients who had undetectable and detectable plasma EBV DNA by qPCR was 87.6% and 60.4%, respectively. The hazard ratio between the two groups was 3.24 (95% CI, 2.40-4.39).

The graph 1200 shows that detectable plasma EBV DNA has a large impact on determining overall survival rates of subjects, including those previously treated for a particular disease. Accordingly, the accuracy of determining the overall survival rates would increase if plasma EBV DNA can be accurately detected from a given biological sample (e.g., post-treatment sample). Although the survival rate for “undetectable” is better than “detectable” for real-time PCR, false negatives occur at a relatively high rate. Thus, a more accurate classification would be desirable. For example, a more sensitive detection of plasma EBV DNA released from residual cancer cells can facilitate the dashed line 1205 being elevated to closer to 100%. Patients in the category of “undetectable” would thus have lower chance of disease recurrence and can be spared from additional treatment.

### III. SEQUENCING FOR DETECTING VIRAL DNA IN POST-TREATMENT SAMPLES

Sequencing techniques have been used as an alternative technique to real-time PCR for cancer screening. For example, in a clinical study involving over 20,000 subjects, it has been shown that plasma EBV DNA analysis by real-time PCR is useful for screening NPC in subjects with no symptoms of NPC (Chan et al. N Engl J Med. 2017; 377:513-522). In the study, subjects with an initial positive test result for plasma EBV DNA were retested 4 weeks later. Subjects with persistently positive results on two occasions were further investigated with nasal endoscopic and MRI examinations. Under this arrangement, 34 NPC patients were identified. The sensitivity and specificity of this NPC screening protocol were 97.1% and 98.6%, respectively. Patients identified by screening had much earlier stage distribution compared those in a historical cohort who did not undergo screening. As a result, the screened subjects had superior progression-free survival with a hazard ratio of 0.1.

The analysis corresponding to the size of the plasma EBV DNA molecules was also performed by next generation sequencing (NGS), which was able to differentiate NPC patients from non-NPC subjects who have detectable plasma EBV DNA by real-time PCR (Lam et al. Proc Natl Acad Sci U S A. 2018; 115:E5115-E5124). Indeed, the specificity of the combined size and count analysis using NGS improved from the 98.6% of the original screening protocol to 99.3% while the sensitivity remained 97.1%. Because of the improvement in specificity, the positive predictive value improved from 11% to 19.6%.

PCR-based techniques are typically optimized for specificity. This can be effective in cancer screening, in which most of the subjects are free from cancer. Such technique, however, would be less effective for predicting relapse in post-treatment samples. This is because such prediction is usually performed over subjects who have been diagnosed of cancer and treated with curative-intent therapies. In this group of patients, the sensitive detection of any residual cancer is desirable so as to allow timely treatment. Thus, achieving high sensitivity can be advantageous for predicting relapse. Despite being capable of performing at high specificity, the PCR-based techniques do not perform well in identifying true positives with high sensitivity. This disclosure investigates whether sequencing can result in higher sensitivity. To this end, sequencing techniques are shown to provide greater accuracy predicting relapse in post-treatment samples.

The sequencing techniques were able to predict relapse in post-treatment samples with substantially higher sensitivity than the real-time PCR. The present techniques thus can include applying sequencing for the analysis of plasma EBV DNA in NPC patients who had received curative-intent cancer treatment. This could be attributed to the fact that the post-treatment samples involve subjects that have or had cancer. As such, accuracy in predicting relapse would increase by maximizing sensitivity.

As mentioned above and described in more detail below, targeted sequencing of the human genome and enrichment of the pathogen genome (e.g., viral genome) can be used to provide a desirable proportion of human and pathogen DNA to analyze, which can provide greater accuracy. The proportion of the human genome covered by the capture probes can be smaller than the proportion of the EBV genome covered by the capture probes. In some instances, the proportion of the human genome covered by the capture probes is adjusted to increase specificity and/or sensitivity when predicting relapse of cancer. As an alternative to target-capture sequencing, whole-genome sequencing or random sequencing can be performed on the post-treatment samples to derive count and size data of the cell-free nucleic acid molecules in the post-treatment samples.

### IV. COUNT-BASED ANALYSIS FOR PREDICTING DISEASE RELAPSE

The sequence reads generated from the targeted sequencing can be aligned and then analyzed to predict relapse of a pathology. For example, a count-based analysis can be performed to determine an amount of sequence reads that align to a viral reference genome. The amount of sequence reads aligning to the viral reference genome can include a proportion of sequence reads aligned to the viral reference genome relative to a total number of sequence reads. In some instances, any function or derivative of a relative amount (abundance) of the viral nucleic acids to human DNA can be used, where examples of a relative amount include a ratio (e.g., proportion) or a difference between the amounts of the viral nucleic acids and human DNA. The determined amount of aligned reads can be compared to a particular cutoff value. If the amount exceeds the cutoff value, disease relapse can be predicted for the given subject.

As shown in FIG. 9, targeted sequencing tends to not work well in differentiating false-positives from early stage NPCs. Therefore, it was initially expected that targeted sequencing would not be appropriate for predicting disease relapse in patients who were already diagnosed with a disease. However, by detecting low levels of EBV DNA in post-treatment samples, targeted sequencing was surprisingly useful in using such data to differentiate patients who have continuous clinical remissions from patients who have disease relapse (e.g., local recurrence, and distant metastasis). Despite the initial skepticism, targeted sequencing demonstrated an unexpected accuracy increase in predicting disease relapse. Furthermore, because of the higher sensitivity and better precision in quantifying EBV DNA in plasma, various thresholds can be adopted for guiding management in different clinical scenarios. For example, two thresholds (high and low) of plasma EBV DNA can be used. For patients who have plasma EBV DNA concentrations higher than the high threshold, preemptive treatment with chemotherapeutic agent can be given to eliminate the concealed cancer cells. For patients who have plasma EBV DNA concentrations between the high and low thresholds, a frequent follow up arrangement can be made to monitor the clinical progress.

For patients with plasma EBV DNA below the lower threshold, less frequent follow-up can be arranged.

### A. Proportion of Sequence Reads

To analyze the cell-free viral DNA in the post-treatment samples, targeted sequencing (e.g., using capture enrichment with specifically designed capture probes) can be used to generate sequence reads. In various implementations, these capture probes can cover the whole EBV genome and multiple genomic regions in the human genome (e.g., including, but not limited to, regions chr1, chr2, chr3, chr5, chr8, chr15 and chr22). Various types of targeted sequencing (e.g., specifically designed capture probes, amplification primers) can be used to enrich for DNA of a viral genome (e.g., across all loci of viral genome or certain loci) relative to the genome of the subject (e.g., of a human genome); such targeted sequencing can be performed to generate sequence reads. After sequencing, the sequenced reads can be mapped to an artificially combined reference sequence, which includes the whole human genome (hg19) and the whole EBV genome. Sequenced reads mapping to unique position in the combined genomic sequence can be used for downstream analysis. The median number of mapped reads is 12.2 million (range: 1.8˜97.6 million).

Mapped reads were then analyzed to determine, for each post-treatment sample, an amount of sequence reads aligning to a viral reference genome corresponding to the virus. The amount of sequence reads can be used to represent the quantity of viral DNA in the post-treatment samples. Using the amount of sequence reads, various types of metrics can be derived to represent the quantity of viral DNA in the each of the post-treatment samples. For example, a metric can include a percentage of viral DNA relative to total DNA in plasma. In another example, the metric can represent a product of the concentration of the total DNA in plasma and the fraction of DNA molecules aligned to the viral reference genome. Other examples of the metric can include: (i) a ratio between the number of viral DNA fragments and the number of non-viral DNA fragments; and (ii) a total number of distinct viral DNA fragments in any sequenced samples.

The amount of sequence reads measured for each post-treatment sample was plotted along the sample's corresponding relapse classification. The classifications included a first classification corresponding to patients who had continuous clinical remission and a second classification corresponding to patients who relapsed. Under the relapse classification, the patients were further divided into the following: (1) a local recurrence (LR) classification which corresponds a local relapse; and (2) a distant metastasis (DM) classification which indicates cancer having metastasized to other organs.

### B. Example

Post-treatment samples from 737 patients treated for nasopharyngeal carcinoma were collected. (Chan et al. J Clin Oncol. 2018; 36: 3091-3100). Each of the post-treatment samples initially had a histologic diagnosis of locoregionally advanced NPC of Union for International Cancer Control (UICC; 6th edition) stage IIB, III, IVA, or IVB, but did not have clinical evidence of persistent locoregional disease or distant metastasis after completion of primary radiation therapy or chemoradiation therapy. In addition, post-treatment venous samples were collected at 6 to 8 weeks after the completion of treatment. The median follow-up interval was 6.6 years. Among the 737 patients, 643 patients (87%) had continuous clinical remission during the first year of the follow-up period, while 94 patients (13%) reported relapse of the disease. Among the 94 relapsed patients, 24 patients (26%) experienced locoregional failure and 70 patients (74%) experienced distant metastasis. Target-capture sequencing was used to identify a proportion of sequence reads for each post-treatment sample.

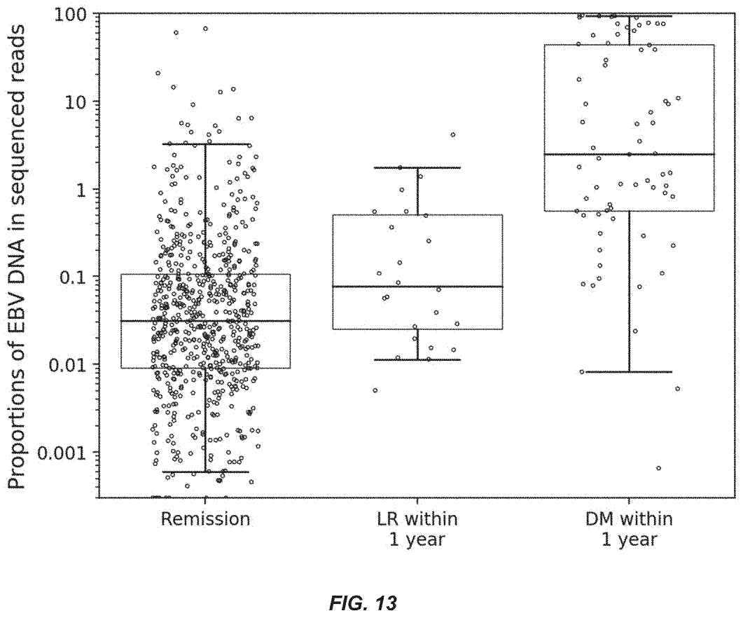

FIG. 13 shows a graph identifying a proportion of sequence reads detected from post-treatment samples using target-capture sequencing. The x-axis represents a classification associated with a post-treatment sample, and the y-axis represents a proportion of plasma EBV DNA reads for the corresponding post-treatment sample. As shown in FIG. 13, percentage of EBV DNA reads over the total number of sequenced reads were significantly higher in the NPC patients who subsequently developed disease relapse compared with those who had continuous remission (p<0.01, Mann-Whitney rank sum test). Among those patients who subsequently developed disease relapse, patients who developed distant metastasis had significantly higher EBV percentage compared with those developed local relapse (p<0.01, Mann-Whitney rank sum test).

Demonstrating substantial improvements over real-time PCR, low levels of EBV DNA were detected in post-treatment samples of patients who had continuous clinical remissions. Similarly, all post-treatment samples under local recurrence and distant metastasis classifications included detectable levels of viral DNA. Moreover, differentiation between relapse and remissions post-treatment samples can be identified. Such findings can be used to identify one or more cutoff values (e.g., 0.01%, 0.1%, 0.45%, 0.5%, 1%).

### C. Method

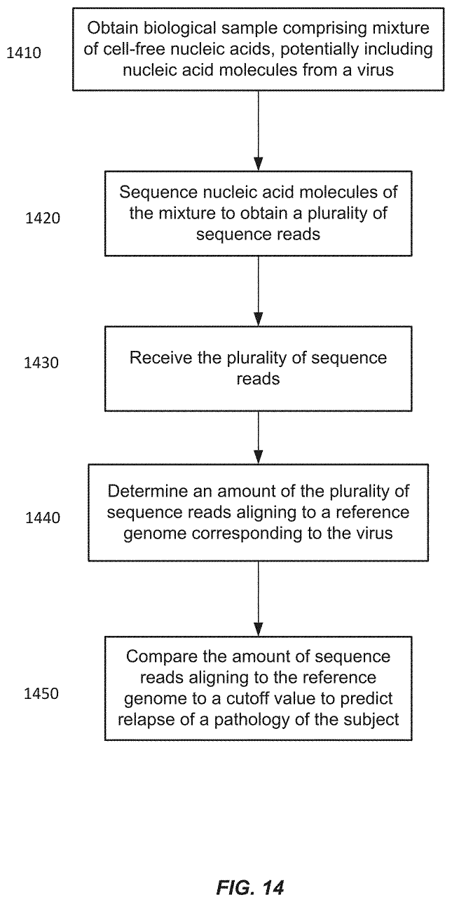

FIG. 14 is a flowchart illustrating a count-based method 1400 using sequence reads of viral nucleic acid fragments in cell-free mixture of a subject to predict disease relapse according to embodiments of the present invention. Aspects of method 1400 can be performed by a computer system, e.g., as described herein.

Method 1400 Can be used to predict disease relapse of a subject previously treated for a pathology and is currently asymptomatic for the pathology. The disease relapse can be predicted from a biological sample of a subject, where the biological sample includes a mixture of cell-free DNA fragments derived from normal tissue (i.e., cells not affected by the pathology) and cell-free DNA fragments (e.g., EBV DNA) derived from diseased tissue that is or has been affected by the pathology (e.g., when the pathology exists in the subject). The cell-free DNA fragment derived from the diseased tissue can be considered clinically-relevant DNA, and the normal tissue can be considered other DNA. In some instances, the pathology corresponds to a cancer caused by a virus (e.g., EBV, HBV, or HPV). The cancer can be one of nasopharyngeal cancer, head and neck squamous cell carcinoma, cervical cancer, and hepatocellular carcinoma.

At block 1410, the biological sample is obtained from the subject. As examples, the biological sample can be blood, plasma, serum, urine, saliva, sweat, tears, and sputum, as well as other examples provided herein. In some embodiments (e.g., for blood), the biological sample can be purified for the mixture of cell-free nucleic acid molecules, e.g., centrifuging blood to obtain plasma.

At block 1420, the mixture of cell-free nucleic acid molecules is sequenced to obtain a plurality of sequence reads. The sequencing may be performed in a variety of ways, e.g., using massively parallel sequencing or next-generation sequencing, using single molecule sequencing, and/or using double- or single-stranded DNA sequencing library preparation protocols. The skilled person will appreciate the variety of sequencing techniques that may be used. As part of the sequencing, it is possible that some of the sequence reads may correspond to cellular nucleic acids.

The sequencing may be target-capture sequencing as described herein. For example, biological sample can be enriched for nucleic acid molecules from the virus. The enriching of the biological sample for nucleic acid molecules from the virus can include using capture probes that bind to a portion of, or an entire genome of, the virus. The biological sample can be enriched for nucleic acid molecules from a portion of a human genome, e.g., regions of autosomes. In other embodiments, the sequencing include random sequencing.

A statistically significant number of cell-free DNA molecules can be analyzed so as to provide an accurate determination of the fractional concentration. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed.

At block 1430, the plurality of sequence reads that were obtained from the sequencing of the mixture of cell free nucleic acid molecules are received. The sequence reads may be received by a computer system, which may be communicably coupled to a sequencing device that performed the sequencing, e.g., via wired or wireless communications or via a detachable memory device.

At block 1440, an amount of the plurality of sequence reads aligning to a viral reference genome corresponding to the virus is determined. Examples of aligning the sequence reads to a viral genome are provided herein. The amount can be determined in a variety of ways based on the number of sequence reads that align to the viral reference genome. For example, the number of sequence reads aligned to the viral reference genome can be normalized. In various embodiments, the normalization may be relative to a volume of the biological sample (or a purified mixture) or relative to a number of sequence reads aligned to a human reference genome.

In some embodiments, the amount of sequence reads aligning to the viral reference genome includes a proportion of sequence reads aligned to the viral reference genome relative to a total number of sequence reads. The total number of sequence reads can be a sum of the sequence reads that aligned to the viral reference genome and the sequence reads that aligned to a human genome. In various implementations, any function or derivative of a relative amount (abundance) of the viral nucleic acids to human DNA can be used, where examples of a relative amount include a ratio (e.g., proportion) or a difference between the amounts of the viral nucleic acids and human DNA.

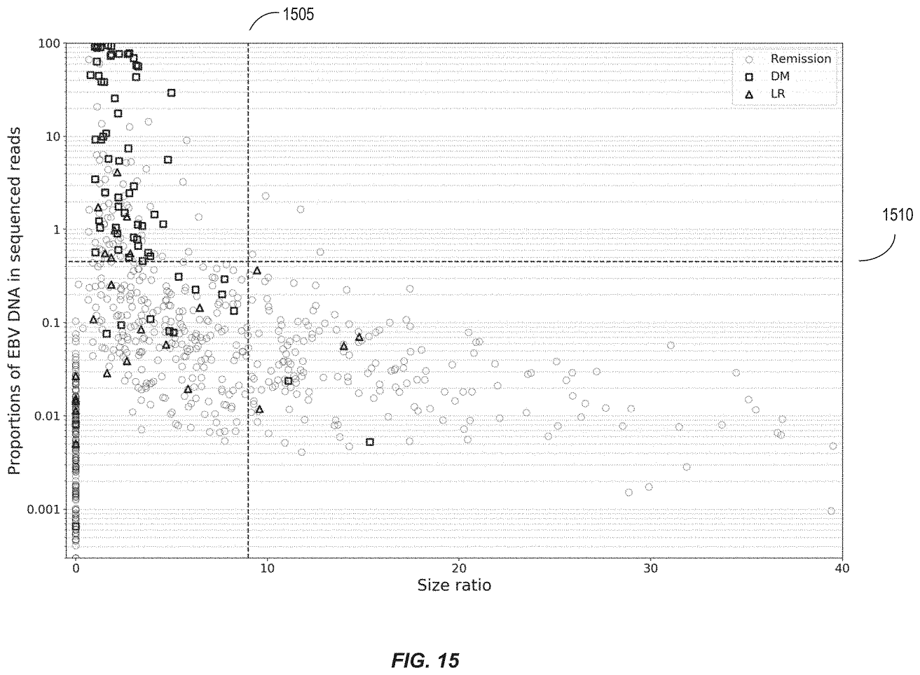

At block 1450, the amount of sequence reads aligning to the viral reference genome is compared to a cutoff value to predict relapse of the pathology for the subject. For example, the cutoff value can be 0.45% for the proportion of plasma EBV DNA reads and can be represented by a horizontal dashed line 1510 of FIG. 15, as shown below. The prediction of relapse can include determining a classification for a relapse of the pathology. The classification can include remission, relapse, loco-regional failure, or distant metastasis.

The cutoff value can be determined from a set of training samples having a known classification of relapse. As examples, the cutoff value can be selected using (1) a value below a lowest amount of sequence reads aligning to the viral reference genome for the training samples classified as having the pathology; (2) a specified number of standard deviations from a mean amount of sequence reads aligning to the viral reference genome for the training samples classified as having the pathology; or (3) a specificity and a sensitivity for determining a correct classification of the training samples.

In some instances, the cutoff value is adjusted to increase sensitivity while compensating for decrease in specificity, or vice versa. For example, the cutoff value for proportion of EBV

DNA can be decreased from 0.45% to 0.1%, to improve sensitivity. In effect, the count-based analyses can identify an optimal sensitivity and specificity for predicting relapse of subjects who had previously completed treatment.

### D. Determining Cutoff Value

Different cutoff values can be selected to optimize the sensitivity and specificity for predicting disease relapse. In some embodiments, the cutoff value can be selected such that a sensitivity of determining the classification of cancer relapse is at least 80% and a specificity of determining the classification of cancer relapse is at least 70%. Additionally or alternatively, cutoff values can be selected to increase sensitivity and specificity for predicting specific types of relapse, including local recurrence and distant metastasis. For example, the cutoff value can be selected such that a sensitivity of determining the classification of local recurrence is at least 50% and a specificity of determining the classification of cancer relapse is at least 70%. In another example, the cutoff value can be selected such that a sensitivity of determining the classification of distant metastasis is at least 80%, and/or a specificity of determining the classification of cancer relapse is at least 70%.

In some embodiments, a cutoff value for the amount of sequence reads aligning to the viral reference genome may be used to determine whether a subject is in remission or has relapsed. For example, subjects who relapsed have a proportion of EBV DNA that are higher than subjects who are in remission or who have detectable plasma EBV DNA from non-cancer cells. In some embodiments, a cutoff value for the proportion of EBV DNA can be about 0.02%, about 0.03%, about 0.04%, about 0.05%, about 0.06%, about 0.07%, about 0.08%, about 0.09%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.75%, about 0.8%, about 0.85%, about 0.9%, about 0.95%, about 1%, or greater than about 1%. For example, the cutoff value can be selected from a range of 0.046% and 0.385% of plasma EBV DNA. In some embodiments, a proportion of EBV DNA at and/or below a cutoff value can be indicative of relapse. In some embodiments, a proportion of EBV DNA at and/or above a cutoff value can be indicative of relapse.

In one embodiment, the cutoff value for the amount of sequence reads can be determined as any value below lowest proportion of the cancer patients being analyzed. In other embodiments, the cutoff values can be determined for example but not limited to the mean size index of the cancer patients minus one standard deviation (SD), mean minus two SD, and mean minus three SD. In yet other embodiments, the cutoff can be determined after the logarithmic transformation of the proportion of plasma DNA fragments mapped to the viral genome, for example but not limited to mean minus one SD, mean minus two SD, mean minus three SD after the logarithmic transformation of the values of the cancer patients. In yet other embodiments, the cutoff can be determined using Receiver Operator Characteristics (ROC) curves or by nonparametric methods, for example but not limited to including 100%, 95%, 90%, 85%, 80% of the patients who relapsed.

### V. COMBINED TECHNIQUES BASED ON COUNT AND SIZE

In addition to the quantity of EBV DNA fragments, we also analyzed the size of the EBV DNA fragments based on the sequencing results of each plasma sample. In this study, we also explored if the size-based analysis (e.g., a size distribution, a size ratio) could enhance the power for predicting disease relapse in NPC patients who had received curative-intent treatment. For example, we looked for differences in the size distributions of plasma viral DNA reads (e.g., EBV, HBV, and HPV) from biological samples of cancer patients who had previously treated for a pathology and are asymptomatic for the pathology. The size distribution of the plasma viral fragments of cancer subjects were statistically significantly shorter than the plasma human DNA fragments in the same subject. The variations in size distributions can be used to identify inter-individual variations in the size profile patterns of sequenced plasma DNA.

In some embodiments, sequencing is used to measures the sizes of the cell-free viral nucleic acids in each sample. For example, the size of each sequenced plasma DNA molecule can be derived from the start and end coordinates of the sequence, where the coordinates can be determined by mapping (aligning) sequence reads to a viral genome. In various embodiments, the start and end coordinates of a DNA molecule can be determined from two paired-end reads or a single read that covers both ends, as may be achieved in single-molecule sequencing. Additionally or alternatively, the size of cell-free viral nucleic acids can be measured in silico or by using a physical process, such as electrophoresis.

In some instances, the size distribution can be displayed as a histogram with the size of a nucleic acid fragment on the horizontal axis. The number of nucleic acid fragments at each size (e.g., within 1 bp resolution) can be determined and plotted on the vertical axis, e.g., as a raw number or frequency percentage. The resolution of size can be more than 1 bp (e.g., 2, 3, 4, or 5 bp resolution). Size distributions (also referred to as size profiles) can be used to determine that the viral DNA fragments in a cell-free mixture from NPC subjects are statistically longer than in subjects with no observable pathology.

### A. Size Ratio

To compare the proportion of plasma viral DNA reads (e.g., EBV reads) within a certain size range (e.g., between 80 and 110 base pairs) among individuals, the amount of plasma viral DNA fragments can be normalized to the amount of autosomal DNA fragments within the same size range. This metric is denoted as a size ratio. A size ratio can be defined by the proportion of plasma viral DNA fragments within a certain size range divided by the proportion of autosomes (e.g., autosomal DNA fragments) within the corresponding size range. For example, a size ratio of EBV DNA fragments between 80 and 110 base pairs would be:

$$Size ratio = \frac{Proportion of EBV DNA within 80 - 110 bp}{P r o p o rtion of human DNA within 80 - 110 bp}$$

The size ratio can indicate the relative proportion of short DNA fragments within each sample. The lower the EBV DNA size ratio, the lower the proportion of EBV DNA molecules of sizes between 80 and 110 bp.

In other embodiments, different size ranges for EBV DNA or human DNA can be used for the calculation of the size ratio. Examples for the lower limit of the size range include but not limited 50 bp, 60 bp, 70 bp, 80 bp, 90 bp, 100 bp, 110 bp, 120 bp. Examples for the upper limit of the size range include but not limited to 60 bp, 70 bp, 80 bp, 90 bp, 100 bp, 110 bp, 120 bp, 130 bp, 140 bp, or 150 bp. In yet other embodiments, more than one size ranges for EBV DNA and/or human DNA can be used. In some embodiments, the size ranges for the EBV DNA and human DNA can be different. In some embodiments, other metrics for measuring the size distribution, for example but not limited to, the number of EBV DNA fragments falling within one or more selected size range(s), mean or median of the size of the EBV DNA fragments, the ratios between the means, medians or modes of the size of the EBV DNA fragments and non-EBV DNA fragments, can be used.

In some embodiments, the size ratios of the sequenced reads are used to determine whether the EBV DNA fragments are from cancer cells or non-cancer cells. Such information can thus be useful to determine whether the detected EBV DNA fragments are indicative of relapse. Various cutoffs can be used to differentiate EBV DNA fragments from human DNA fragments, and/or cancerous EBV DNA fragments from non-cancerous EBV DNA fragments. The amount of EBV DNA fragments that are below the cutoff values can be measured to classify whether a corresponding subject is under continuous clinical remission or relapse (e.g., local recurrence, distant metastasis).

Other embodiments can use other statistical values of a size distribution (e.g., an average, median, mode, ratio of amount of reads at one size range relative to another size range) of sequence reads in a cell-free sample that align to the EBV genome to predict disease relapse.

### B. Example

Post-treatment samples from 737 patients treated for nasopharyngeal carcinoma were collected. (Chan et al. J Clin Oncol. 2018; 36: 3091-3100). Each of the post-treatment samples initially had a histologic diagnosis of locoregionally advanced NPC of Union for International Cancer Control (UICC; 6th edition) stage IIB, III, IVA, or IVB, but did not have clinical evidence of persistent locoregional disease or distant metastasis after completion of primary radiation therapy or chemoradiation therapy. In addition, post-treatment venous samples were collected at 6 to 8 weeks after the completion of treatment. The median follow-up interval was 6.6 years. Among the 737 patients, 643 patients (87%) had continuous clinical remission during the first year of the follow-up period, while 94 patients (13%) reported relapse of the disease. Among the 94 relapsed patients, 24 patients (26%) experienced locoregional failure and70 patients (74%) experienced distant metastasis. Target-capture sequencing was used to assess size of cell-free viral nucleic acids in each post-treatment sample.

FIG. 15 shows a graph identifying proportion and size ratio of EBV DNA detected from post-treatment samples using target-capture sequencing. The x-axis represents a EBV DNA size ratio within the 80-110 bp size range of a post-treatment sample, and the y-axis represents a proportion of plasma EBV DNA reads for the corresponding post-treatment sample. Each of the post-treatment samples was identified as under remission represented by a circle shape, distant metastasis (DM) represented by a square shape, or loco-regional failure (LR) represented by a triangle shape. As an illustrative example of using cutoffs for predicting relapse, FIG. 15 shows a vertical dashed line 1505 which represents a cutoff value of 9 for the size ratio and a horizontal dashed line 1510 which represents a cutoff value of 0.45% for the proportion of plasma EBV DNA reads. Sequence reads having size ratio below the 9 cutoff value and proportion of EBV DNA above the 0.45% cutoff value were used to predict that a corresponding subject had relapsed.

As shown by in FIG. 15, the circles and square shapes under the cutoff criteria above indicate that 63 (67%) of the 94 patients subsequently develop disease relapse. For the 70 subjects who would subsequently develop distant metastasis, 56 (80%) were identified. For the 24 subjects who would subsequently develop local relapse, 7 (29%) were identified. Of the 643 patients who would be in continuous remission, 583 (91%) were outside of the quantity cutoff value (e.g., 0.45% EBV DNA proportion represented by the horizontal dashed line 1510) and size cutoff value (e.g., 9 size ratio represented by the vertical dashed line 1505) for EBV DNA.

Thus, the combined analysis using target-capture sequencing had a higher combination of sensitivity and specificity (67% and 91% respectively) than real-time PCR (70% and 79% respectively) for predicting post-treatment samples that would subsequently relapse within the first year after the completion of treatment. This shows that the combined analysis is substantially more effective in predicting relapse in post-treatment samples over real-time PCR.

### C. Method

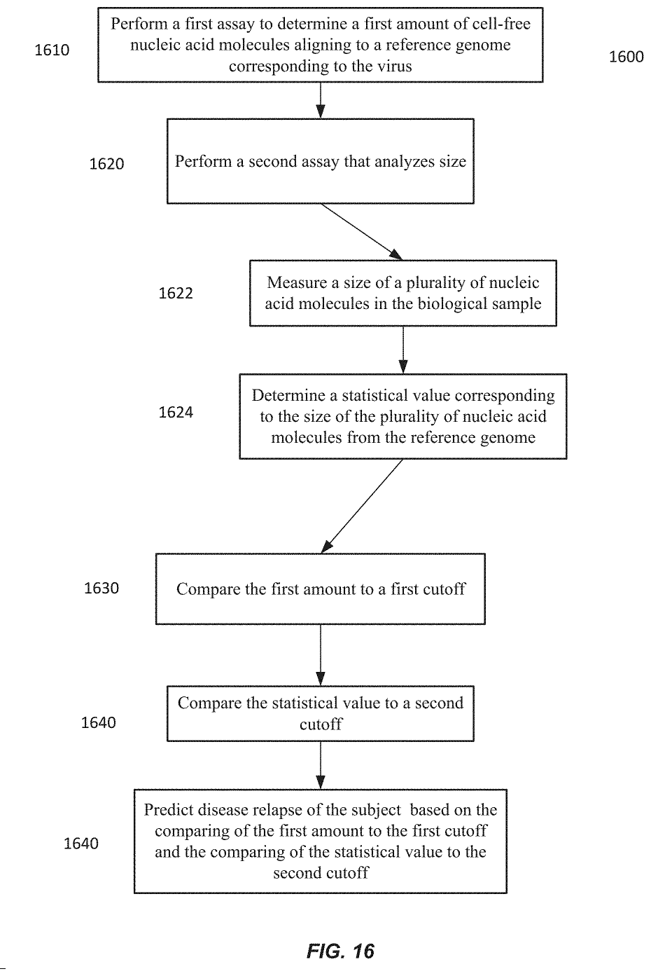

FIG. 16 is a flowchart for a method that combined a count-based and a size-based analysis of viral nucleic acid fragments to predict disease relapse according to embodiments of the present invention. At least a portion of the method may be performed by a computer system.

Method 1600 can analyze a biological sample to predict relapse of a biological sample from a subject previously treated for a pathology and is currently asymptomatic for the pathology. The disease relapse can be predicted from a biological sample of a subject, where the biological sample includes a mixture of cell-free DNA fragments derived from normal tissue (i.e., cells not affected by the pathology) and cell-free DNA fragments (e.g., EBV DNA) derived from diseased tissue that is or has been affected by the pathology (e.g., when the pathology exists in the subject).

The cell-free DNA fragment derived from the diseased tissue can be considered clinically-relevant DNA, and the normal tissue can be considered other DNA. In some instances, the pathology corresponds to a cancer caused by a virus (e.g., EBV, HBV, or HPV). The cancer can be one of nasopharyngeal cancer, head and neck squamous cell carcinoma, cervical cancer, and hepatocellular carcinoma.

At block 1610, a first assay is performed. The first assay can analyze a plurality of cell-free nucleic acid molecules from a first biological sample of the subject to determine a first amount of the plurality of cell-free nucleic acid molecules aligning to a viral reference genome corresponding to the virus. As examples, the first assay can include sequencing, e.g., as performed in method 1400. Other examples are real-time PCR or digital PCR.

At block 1620, a second assay is performed using a size-based analysis. Blocks 1622 and 1624 can be performed as part of performing the second assay. The second assay can be performed on a second biological sample, which may be the same or different than the first biological sample. The first biological sample and the second biological sample can be from a same blood sample (e.g., different plasma/serum portions). In some embodiments, the second assay is only performed when the first amount is above the first cutoff.

At block 1622, a size of each of the plurality of nucleic acid molecules in a second biological sample is measured. The size may be measured via any suitable method, for example, methods described above. As examples, the measured size can be a length, a molecular mass, or a measured parameter that is proportional to the length.

In some embodiments, both ends of a nucleic acid molecule can be sequenced and aligned to a genome to determine starting and ending coordinates of the nucleic acid molecule, thereby obtaining a length in bases, which is an example of size. Such sequencing can be target-capture sequencing, e.g., involving capture probes as described herein. Other example techniques for determining size include electrophoresis, optical methods, fluorescence-based method, probe-based methods, digital PCR, rolling circle amplification, mass spectrometry, melting analysis (or melting curve analysis), molecular sieving, etc. As an example for mass spectrometry, a longer molecule would have a larger mass (an example of a size value).

At block 1624, a statistical value corresponding to the size of the plurality of nucleic acid molecules from the viral reference genome is determined. In some embodiments, the statistical value can include a size ratio between: (1) a first proportion of sequence reads of nucleic acid molecules that align to the viral reference genome with the size within a given range; and (2) a second proportion of sequence reads of nucleic acid molecules that align to a human reference genome with the size within the given range. In various embodiments, the given range can be about 80 to about 110 base pairs, about 50 to about 75 base pairs, about 60 to about 90 base pairs, about 90 to about 120 base pairs, about 120 to about 150 base pairs, or about 150 to about 180 base pairs. In other embodiments, the statistical value can be an inverse of the size ratio, thereby using a size index.

The statistical value can correspond to a size distribution of the plurality of nucleic acid molecules from the viral reference genome. A cumulative frequency of fragments smaller than a size threshold is an example of a statistical value. The statistical value can provide a measure of the overall size distribution, e.g., an amount of small fragments relative to an amount of large fragments. As another example, the statistical value can include a ratio of: (1) a first amount the plurality of nucleic acid molecules in the biological sample from the viral reference genome that are within a first size range; and (2) a second amount the plurality of nucleic acid molecules in the biological sample from the viral reference genome that are within a second size range that is different than the first size range. For example, the first range could be fragments below a first size threshold and the second size range could be fragments above a second size threshold. The two ranges can overlap, e.g., when the second size range is all sizes.

In various embodiments, the statistical value can be an average, mode, median, or mean of the size distribution. In other embodiments, the statistical value can be a percentage of the plurality of nucleic acid molecules in the biological sample from the viral reference genome that are below a size threshold (e.g., 150 bp). For such a statistical value, the subject can be determined to be positive for the pathology when statistical value is below the cutoff value.

In some embodiments, the statistical value can be normalized using an amount of cell-free nucleic acid molecules having a size within a different range and aligning to the viral reference genome. As another example, the statistical value can be normalized using an amount of cell-free nucleic acid molecules having a size within the given range and aligning to an autosomal genome.

At block 1630, the first amount is compared to a first cutoff. It can be determined whether the first amount exceeds the first cutoff (e.g., above). For example, the first cutoff can be 0.45% for the proportion of plasma EBV DNA reads and can be represented by the horizontal dashed line 1510 of FIG. 15. An extent that the first amount exceeds the first cutoff can be determined, e.g., so as to inform the final determination of disease relapse.

At block 1640, the statistical value is compared to a second cutoff. It can be determined whether the statistical value exceeds the second cutoff (e.g., above or below, depending on how the second amount is defined). For example, the second cutoff can be 9 size ratio between EBV DNA and human DNA, and can be represented by the vertical dashed line 1505 of FIG. 15. An extent that the second amount exceeds the second cutoff can be determined, e.g., so as to inform the final determination of the disease relapse.

At block 1650, a classification for a relapse of the pathology is determined based on the comparing of the first amount to the first cutoff and the comparing of the statistical value to the second cutoff. In some embodiments, the subject is determined to have relapsed only if the first amount exceeds the first cutoff (e.g., 0.45% EBV DNA proportion represented by the horizontal dashed line 1510 of FIG. 15) and the second amount exceeds the second cutoff (e.g., 9 size ratio represented by the vertical dashed line 1505 of FIG. 15).

Different first and second cutoff values can be selected to increase the sensitivity and/or specificity for predicting disease relapse. In some embodiments, each of the first and second cutoff values can be selected such that a sensitivity of determining the classification of cancer relapse is at least 80% and a specificity of determining the classification of cancer relapse is at least 70%. Additionally or alternatively, first and/or second cutoff values can be selected to increase sensitivity and specificity for predicting specific types of relapse, including local recurrence and distant metastasis. For example, each of the first and second cutoff values can be selected such that a sensitivity of determining the classification of local recurrence is at least 50% and a specificity of determining the classification of cancer relapse is at least 70%. In another example, each of the first and second cutoff values can be selected such that a sensitivity of determining the classification of distant metastasis is at least 80%, and/or a specificity of determining the classification of cancer relapse is at least 70%.

In some instances, each of the first and second cutoff values can be adjusted to increase specificity while compensating for slight decrease in sensitivity, or vice versa. In effect, the combined analyses can identify an optimal sensitivity and specificity for predicting disease relapse of subjects who had completed treatment.

### D. Determining Cutoff Value for Size

In some embodiments, a cutoff value for the size (e.g., size ratio, size distribution) may be used to determine whether a subject is in remission or has relapsed. For example, subjects who relapsed have a lower size ratio within the size range of 80 to 110 bp than subjects who are in remission or who have detectable plasma EBV DNA from non-cancer cells. In some embodiments, a cutoff value for a size ratio can be about 0.1, about 0.5, about 1, about 2, about 3, about 4, about 5, about 6, about 7, about 8, about 9, about 10, about 11, about 12, about 13, about 14, about 15, about 16, about 17, about 18, about 19, about 20, about 25, about 50, about 100, or greater than about 100. For example, the cutoff value can be selected from a size ratio between 6 and 11. In some embodiments, a size ratio at and/or below a cutoff value can be indicative of relapse. In some embodiments, a size ratio at and/or above a cutoff value can be indicative of relapse.

In some embodiments, a cutoff value for a size index can be about or least 10, about or least 2, about or least 1, about or least 0.5, about or least 0.333, about or least 0.25, about or least 0.2, about or least 0.167, about or least 0.143, about or least 0.125, about or least 0.111, about or least 0.1, about or least 0.091, about or least 0.083, about or least 0.077, about or least 0.071, about or least 0.067, about or least 0.063, about or least 0.059, about or least 0.056, about or least 0.053, about or least 0.05, about or least 0.04, about or least 0.02, about or least 0.001, or less than about 0.001. In some embodiments, a size index at and/or below a cutoff value can be indicative of relapse. In some embodiments, a size index at and/or above a cutoff value can be indicative of relapse.

In one embodiment, the cutoff value for the size ratio or size index can be determined as any value below lowest proportion of the cancer patients being analyzed. In other embodiments, the cutoff values can be determined for example but not limited to the mean size index of the cancer patients minus one standard deviation (SD), mean minus two SD, and mean minus three SD. In yet other embodiments, the cutoff can be determined after the logarithmic transformation of the proportion of plasma DNA fragments mapped to the viral genome, for example but not limited to mean minus one SD, mean minus two SD, mean minus three SD after the logarithmic transformation of the values of the cancer patients. In yet other embodiments, the cutoff can be determined using Receiver Operator Characteristics (ROC) curves or by nonparametric methods, for example but not limited to including 100%, 95%, 90%, 85%, 80% of the patients who relapsed.

### VI. COMPARISON BETWEEN REAL-TIME PCR AND SEQUENCING FOR PREDICTING DISEASE RELAPSE

We compared the diagnostic performances of the count-based analysis, combined count- and size-based analysis, and real-time PCR in predicting disease relapse that occurred within 1-year post treatment in subjects, by using receiver operating characteristic (ROC) curve analysis. In addition, performances of the count-based analysis and real-time PCR were also compared with respect to predicting overall survival rates of patients, in which the survival rates were predicted based on detected amount of viral DNA.

### A. Predicting Disease Relapse

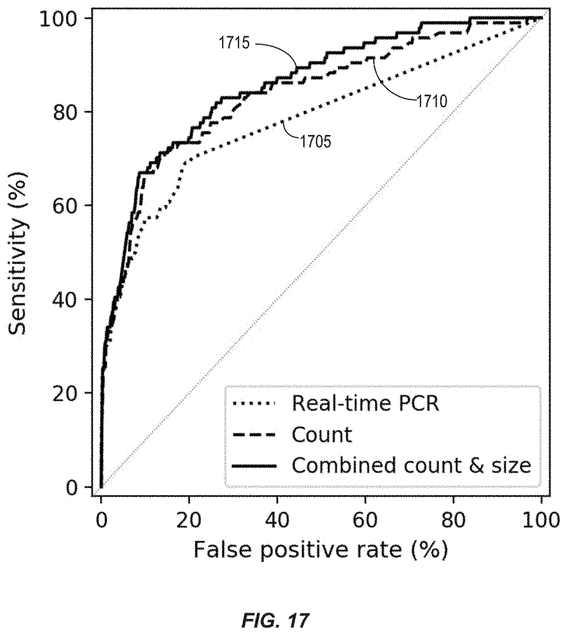

FIG. 17 shows receiver operating characteristic (ROC) curve for predicting disease relapse based on the analysis of plasma samples collected at 6 weeks after completion of curative-intent treatment for NPC patients. The ROC curves include a dotted line 1705 that represents real-time PCR, a dashed line 1710 that represents the count-based analysis, and a solid line 1715 that represents the combined count- and size-based analysis. The combined analysis was performed using target-capture sequencing with probes designed to capture the entire EBV genome. The real-time PCR performance is based on quantitative plasma EBV DNA values determined by the real-time PCR assay to differentiate relapsed subjects from those in remission.

The area under curve (AUC) values were 0.78 and 0.84 for real-time PCR and count-based analysis, respectively. The AUC values improved even further when size-based analysis was combined into the count-based analysis. With the combination of size ratio, the AUC was 0.86, which was significantly greater (p<0.01, bootstrap test) than the AUC of the real-time PCR.

In another example, specificity and sensitivity across various techniques were determined for further evaluation. In this example, diagnostic performances of the count-based analysis with different EBV DNA amount cutoffs, combined count- and size-based analysis with various EBV DNA amount cutoffs (e.g., 0.1%, 0.45%) and size ratio cutoff (e.g., 9), and real-time PCR were evaluated. The results are shown in Table 2 as follows:

TABLE 2
Sensitivities and specificities for predicting
local relapse and distant metastasis.

| Outcome | Parameter used | Sensitivity (%) | Specificity (%) |
|---|---|---|---|
| All | Real-time PCR | 70.2 | 78.7 |
| relapse | EBV% ≥0.1 | 77.7 | 72.8 |
| | EBV% ≥0.45 | 67.0 | 90.0 |
| | EBV% ≥0.1 + | 76.6 | 76.4 |
| | size ratio ≤9 | | |
| | EBV% ≥0.45 + | 67.0 | 90.7 |
| | size ratio ≤9 | | |
| Distant | Real-time PCR | 82.9 | 78.7 |
| metastasis | EBV% ≥0.1 | 88.6 | 72.8 |
| | EBV% ≥0.45 | 80.0 | 90.0 |
| | EBV% ≥0.1 + | 88.6 | 76.4 |
| | size ratio ≤9 | | |
| | EBV% ≥0.45 + | 80.0 | 90.7 |
| | size ratio ≤9 | | |
| Local | Real-time PCR | 33.3 | 78.7 |
| relapse | EBV% ≥0.1 | 45.8 | 72.8 |
| | EBV% ≥0.45 | 29.2 | 90.0 |
| | EBV% ≥0.1 + | 41.7 | 76.4 |
| | size ratio ≤9 | | |
| | EBV% ≥0.45 + | 29.2 | 90.7 |
| | size ratio ≤9 | | |

As shown in Table 2, the count-based and combined analyses are respectively associated with higher combinations of sensitivity and specificity than real-time PCR in predicting disease relapse that occurred within the first-year post treatment. Since predicting relapse involves post-treatment samples having low levels of viral DNA, identifying true positives (TP) of relapse is advantageous. Thus, the count-based and combined analyses would be the appropriate techniques over real-time PCR.

### B. Survival Rates Based on Count- and Size-Based Analysis

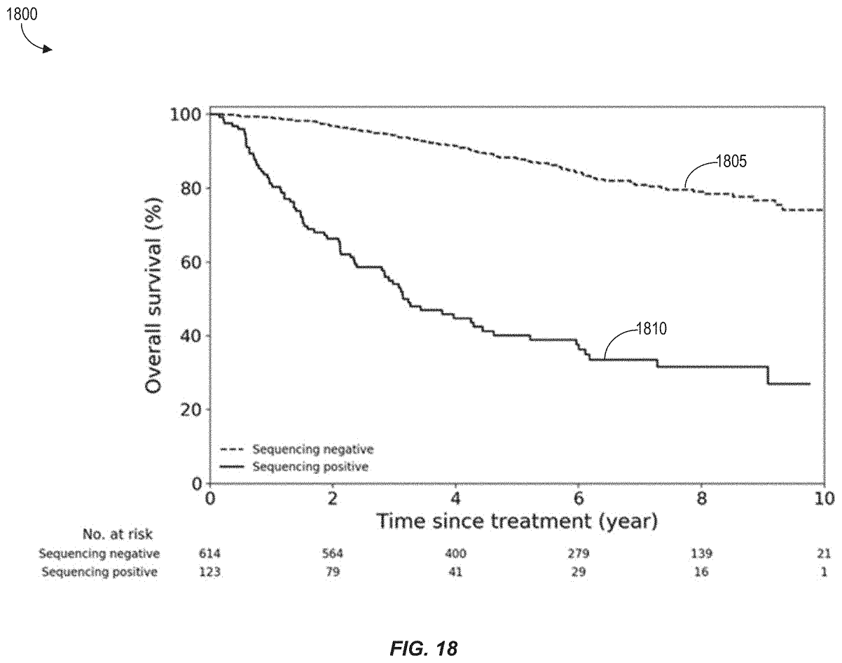

The cutoffs described above can be used to classify patients into different prognostic groups. FIG. 18 shows a graph identifying overall survival rates of NPC patients grouped according to the combined analysis using targeted-capture sequencing. Similar to the cutoffs used in FIG. 15, 0.45% for proportion of EBV DNA and 9 for size ratio were used. Subjects who satisfied both cutoffs were defined as “sequencing positive” and represented by dashed line 1805, and subjects who satisfied only one or none of the cutoffs were defined as “sequencing negative” and represented by solid line 1810. As shown in FIG. 18, patients classified as sequencing positive were associated with superior overall survival compared to those classified as sequencing negative (p<0.0001, log-rank test). In particular, the 5-year survival of patients who are sequencing positive and sequencing negative were 88.0% and 40.1%, respectively. The hazard ratio between the two groups was 6.44 (95% CI, 4.74-8.74). Compared to the overall survival rates shown in FIG. 12 that used real-time PCR, the survival rates shown in FIG. 18 showed a greater separation between the sequencing-positive subjects and sequencing-negative subjects. This may indicate that targeted-capture sequencing is more likely to detect those actually having a disease relapse that corresponds to a lower survival rate.

Cox Proportional Hazards model was used to evaluate the prognostic power of different variables, including the combined analysis. For real-time PCR, the hazard of patients who had undetectable plasma EBV DNA was compared with the hazard of patients who had detectable plasma EBV DNA. For count-and-size analysis, the hazard of patients whose proportion of EBV DNA fulfilled the cutoff 0.45% and size ratio fulfilled the cutoff 9 was compared with the hazard of patients who fulfilled only one or none of the cutoffs. For comparison, a cancer-level classification (e.g., UICC overall stage), treatment modality, size of the tumor (e.g., T stage), spread of cancer to nearby lymph nodes (e.g., N stage), age, and sex were other variables that were also included in the model. Univariate analysis for overall survival suggested that the combined analysis was the most significant independent prognostic factor (p<0.0001), followed by real-time PCR (p<0.0001), UICC overall stage (p<0.0001), treatment modality (p<0.0001), T stage (p=0.0001), N stage (p=0.0004), age (p=0.013). Sex was a marginally significant factor (p=0.051). On a multivariate Cox Proportional Hazards model, the combined analysis remained the most powerful independent prognostic factor for overall survival (p<0.0001), followed by T stage (p=0.02), sex (p=0.02), whereas UICC overall stage (p=0.05), real-time PCR (p=0.58), treatment modality (p=0.96), N stage (p=0.45), and age (p=0.07) were not.

The combined analysis of viral DNA can be used to estimate the proportion of viral DNA in various biological samples, including post-treatment samples. The proportion of viral DNA can then be used to facilitate prediction of the subject's overall survival rate. For example, patients were stratified into different prognostic groups by each 10-fold increase in proportion of EBV DNA, to determine whether overall survival rates correlate to amount of EBV DNA.

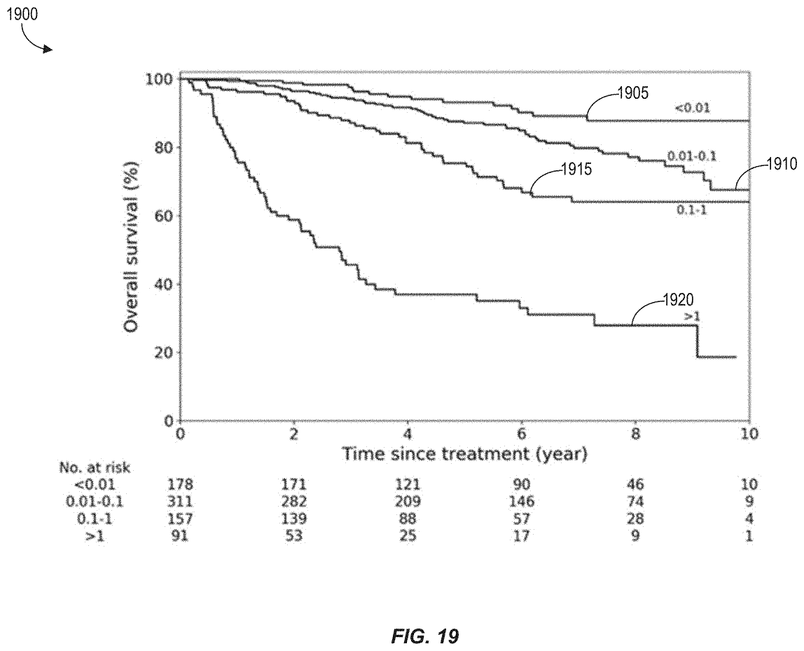

FIG. 19 shows a graph 1900 identifying overall survival rates of NPC patients grouped according to their estimated sequencing-based EBV DNA levels. Four prognostic groups were classified, including a first group of patients with proportions of EBV DNA below 0.01% (as represented by line 1905), a second group of patients with proportions of EBV DNA within 0.01%-0.1% (as represented by line 1910), a third group of patients with proportions of EBV DNA within 0.1%-1% (as represented by line 1915), and a fourth group of patients with proportions of EBV DNA higher than 1%, respectively (as represented by line 1920). As shown in FIG. 19, a worsening overall survival was observed with increasing proportion of EBV DNA in plasma. When compared to the patients with proportions of EBV DNA below 0.01%, the other patient groups with proportions of EBV DNA of 0.01%-0.01, 0.1%-1%, or >1% had the hazard ratio (HR) values of 2.12, 3.74, 13.56, respectively (see Table 3).

TABLE 3
Correlation of plasma EBV DNA level determined by sequencing
with overall survival among 737 analyzed subjects

| | | Overall Survival | | | |
|---|---|---|---|---|---|
| Proportions of | No. of | No. of | 5-Year | HR | |
| EBV DNA (%) | patients | event (%) | Rate, % | (95% CI) | p-value |
| <0.01 | 178 | 15 (8.4) | 93.2 | 1 | — |
| 0.01-0.1 | 311 | 53 (17.0) | 87.1 | 2.12 | 0.01 |
| | | | | (1.20-3.76) | |
| 0.1-1 | 157 | 42 (26.8) | 75.4 | 3.74 | <0.0001 |
| | | | | (2.07-6.74) | |
| >1 | 91 | 59 (64.8) | 37.0 | 13.56 | <0.0001 |
| | | | | (7.65-24.04) | |

Thus, the count-based analysis can be implemented for modeling overall survival rates corresponding 10-fold increments of proportion of EBV DNA. Across various evaluations, the results from the combined analysis emerged as the most significant independent prognostic factor. For univariate analysis, the combined analysis was the most significant prognostic factor for overall survival (p<0.0001), followed by real-time PCR (p<0.0001), UICC overall stage (p<0.0001), treatment modality (p<0.0001), T stage (p=0.0001), N stage (p=0.0004), and age (p=0.013). In contrast, sex was marginally significant (p=0.051) on univariate analysis. For multivariate analysis, the combined analysis still remained the most powerful independent prognostic factor for overall survival (p<0.0001), followed by T stage (p=0.02), sex (p=0.03),

UICC overall stage (p=0.04). In contrast, real-time PCR (p=0.41), treatment modality (p=0.77), N stage (p=0.33), and age (p=0.07) were not considered as effective prognostic factors for multivariate analysis.

### C. Predicting Patients with High Survival Rates

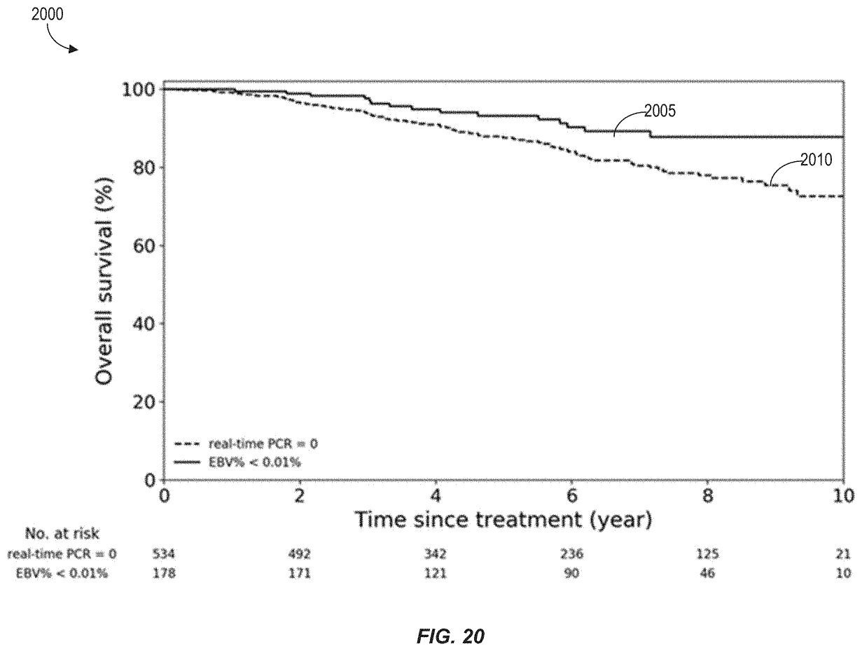

In addition, the count-based analysis was able to predict a group of patients associated with high overall survival rates. The prediction can be based on identifying one or more patients for which very low amount of viral DNA was detected. FIG. 20 shows a graph 2000 identifying overall survival rates of NPC patients who had proportions of EBV DNA in plasma below 0.01%. In the graph 2000, a line 2005 represented survival rates of NPC patients with less than 0.01% EBV DNA detected using count-based analysis, and a line 2010 represented survival rates of NPC patients with no EBV DNA being detected using real-time PCR. For patients who had proportion of EBV DNA below 0.01% using the count-based analysis, their 5-year survival rate was 93.2%. In contrast, the 5-year survival rate of patients who had with undetectable plasma EBV DNA using qPCR only achieved 87.6%. Indeed, as shown in FIG. 2000, the overall survival of patients with proportion of EBV DNA using count-based analysis was significantly better than patients with undetectable plasma EBV DNA by qPCR (p=0.01, log-rank test). As such, count-based analysis demonstrate superiority of identifying patients with good prognosis, which can be also effective in predicting disease relapse in post-treatment samples.

### VII. SEQUENCING VIRAL DNA TO IDENTIFY PREGNANCY ASSOCIATED ABNORMALITIES

Although viral-related diseases such as NPC is most prevalent between the age of 40 to 60 years, such disease can also occur in the younger age group during their reproductive age. The occurrence of NPC during pregnancy can have significant adverse impact both physically and psychologically to pregnant women. In this regard, we explored if count-based and combined analyses of viral DNA (e.g., EBV, HPV) would be more accurate than real-time PCR for identifying NPC and other pregnancy associated abnormalities in pregnant women. We analyzed samples from 26 NPC patients and 26 pregnant women who did not have NPC. Real-time PCR for quantitative analysis for plasma EBV DNA and target-capture sequencing of plasma EBV DNA as described above were performed.

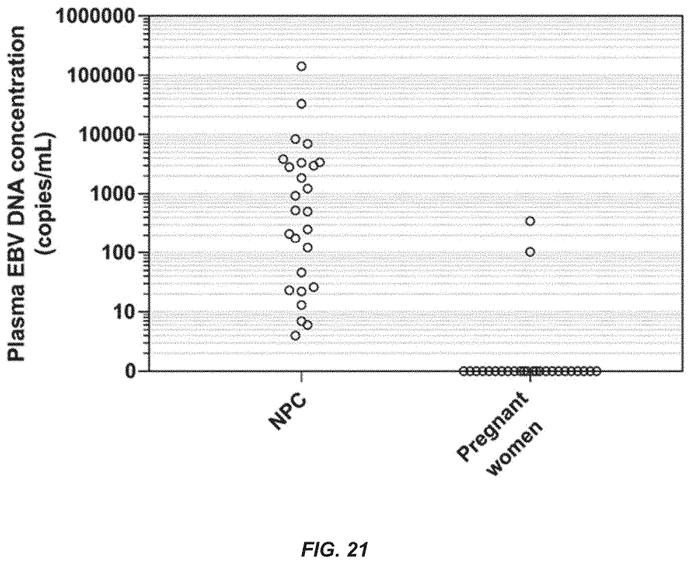

FIG. 21 shows plasma EBV DNA concentration by real-time PCR across NPC patients and pregnant women for which NPC was not identified. The x-axis represents a classification corresponding to a subject (e.g., NPC, pregnant women), and the y-axis represents viral DNA concentration for the biological sample corresponding to the subject. As shown in FIG. 21, plasma

EBV DNA was detectable in all the 26 NPC patients. However, 2 (8%) of the 26 pregnant women had detectable plasma EBV DNA concentration ranging between 100-1000 copies per mL. Thus, if detectable plasma EBV DNA is used as a screening test for NPC in pregnant women, the sensitivity and specificity of the test were 100% and 92%, respectively. Other than the presence of EBV DNA, other types of separation between NPC and pregnancy women for the EBV DNA do not appear available, due to significant viral DNA being detected in two pregnancies.

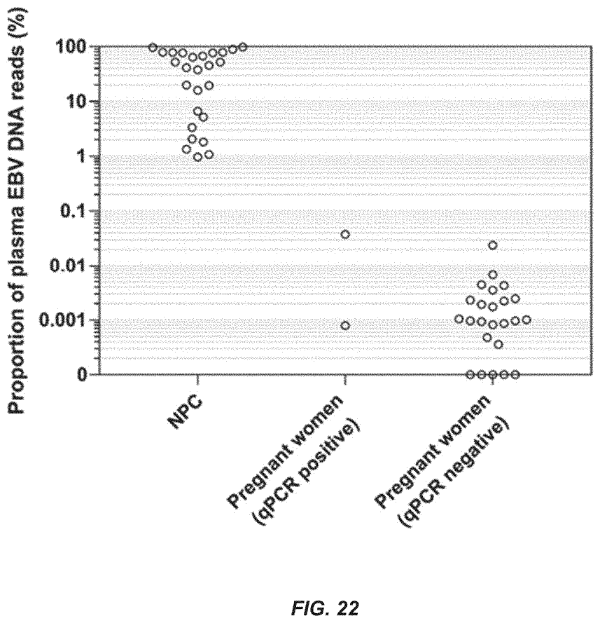

FIG. 22 shows plasma EBV DNA concentration using count-based analysis across NPC patients and pregnant women for which NPC was not identified. The x-axis represents a classification corresponding to a subject (e.g., NPC, pregnant women), and the y-axis represents viral DNA concentration for the biological sample corresponding to the subject. The group of pregnant subjects were further divided into two sub-groups, qPCR positive and qPCR negative. Target-capture sequencing was used to determine the plasma EBV DNA concentration.

Using a cutoff of >0.1% for EBV DNA proportion, all of the 26 NPC patients were correctly identified as positive and all 26 pregnant women were identified as negative, regardless of their EBV DNA status by real-time PCR. Hence, the count-base analysis performed more accurately than the real-time PCR analysis, with 100% sensitivity and 100% specificity. Further, in contrast to real-time PCR, there was a clearer separation of detected EBV DNA in count-based analysis using target-capture sequencing, which facilitates selection of a cutoff value that can accurately identify a level of virus-related pathology.

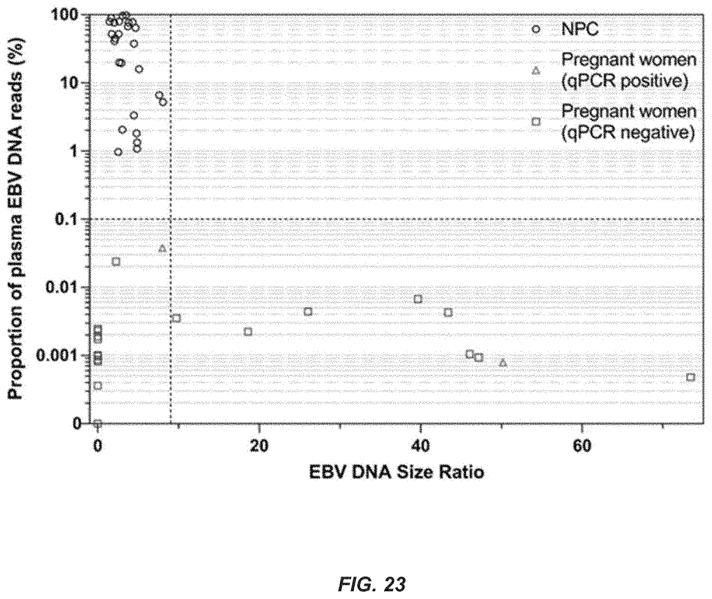

FIG. 23 shows count- and size-based analysis of plasma EBV DNA across NPC patients and pregnant women for which NPC was not identified. The x-axis represents a classification corresponding to a subject (e.g., NPC, pregnant women), and the y-axis represents viral DNA concentration for the biological sample corresponding to the subject. The group of pregnant subjects were further divided into two sub-groups, qPCR positive and qPCR negative. Target-capture sequencing was used to determine the plasma EBV DNA concentration.

In FIG. 23, a first cutoff of 0.1% for EBV DNA proportion and a second cutoff of 9.0 for size ratio were used. Similar to the examples in FIG. 23, all the 26 NPC patients were classified as positive, and all 26 pregnant women were classified as negative. Accordingly, the sensitivity and specificity of using count- and size-based analysis for screening NPC in pregnant women would have 100% sensitivity and 100% specificity. Similar to count-based analysis, there was a clearer separation of detected EBV DNA in count- and size-based analysis using target-capture sequencing, which facilitates selection of a cutoff value that can accurately identify a level of virus-related pathology.

### VIII. EXAMPLES SEQUENCING TECHNIQUES

Various example techniques are described below, which may be implemented in various embodiments.

### A. Sample Collection

Blood samples of NPC patients were collected at 6 to 8 weeks after the completion of treatment. NPC patients were recruited from six oncology centers in Hong Kong with informed consents. Eligible patients were those whose ages were 18 years or older, with a histologic diagnosis of locoregionally advanced NPC of Union for International Cancer Control (UICC; 6th edition) stage IIB, III, IVA, or IVB. The eligible patients showed no clinical evidence of persistent locoregional disease or distant metastasis after completion of primary radiation therapy or chemoradiation therapy. In some instances, for each post-treatment plasma sample analyzed, DNA was extracted using the QIAamp Circulating Nucleic Acid kit.

Regarding DNA library construction, indexed plasma DNA libraries can be constructed using the TruSeq Nano library preparation kit according to the manufacturer's protocol. The adaptor-ligated DNA was amplified with 8 cycles of PCR using the TruSeq Nano PCR amplification kit (Illumina). The amplification products were captured using the myBaits Custom Capture Panel (Arbor Biosciences) using the custom-designed probes covering the viral and human genomic regions stated above. After target capturing, 14 cycles of PCR amplification were performed and the products were sequenced using the Illumina NextSeq platform. For each sequencing run, 24 samples with unique sample barcodes were sequenced using the paired-end mode.

Regarding sequencing of DNA libraries, the multiplexed DNA libraries can be sequenced using either the NextSeq 500 (Illumina). A paired-end sequencing protocol was used, with 75 nucleotides being sequenced from each end.

Regarding alignment of sequencing data, the paired-end sequencing data can be analyzed by means of the SOAP2 in the paired-end mode. The paired-end reads were aligned to the combined reference genomes including reference human genome (hg19) and EBV genome (AJ50779.2). Up to two nucleotide mismatches were allowed for the alignment of each end. Only paired-end reads with both ends uniquely aligned to the same chromosome with the correct orientation, spanning an insert size within 600 bp, were used for downstream analysis.

In some embodiments, sequencing data analysis was performed by bioinformatics programs written in Perl and R languages. The Kruskal-Wallis test was used to compare the plasma

EBV DNA concentrations among the NPC patients, non-cancer subjects with transiently positive EBV DNA and non-cancer subjects with persistently positive EBV DNA in the whole screening cohort and in the exploratory and validation datasets. The Kruskal-Wallis test was also used to compare the proportion of EBV DNA reads in the three groups in the exploratory and validation datasets. A P value of <0.05 was considered as statistically significant.

### B. Capture Probes

The specificity and/or sensitivity of detecting tumor-derived nucleic acids can be proportional with the concentration of tumor-derived nucleic acids in the sample. Accordingly, target-specific enrichment can be used to increase the concentration of tumor-derived nucleic acids in the sample. For example, a DNA probe having a sequence complementary to, and capable of binding, BamHI-W sequence in EBV DNA can be used to perform targeted enrichment of the EBV DNA fragments in the sample. The DNA probe is also labeled with a high affinity tag (e.g., biotin), which allows the target-bound probe to be recovered. Following recovery of the target-bound probe, the EBV DNA is dissociated and separated from the probe. Subsequently, the enriched sample can be analyzed according the methods described herein.

For enrichment of viral DNA molecules from the plasma DNA samples for subsequent sequencing analysis, target enrichment with EBV capture probes was performed. The EBV capture probes which covered the entire EBV genome were ordered from Arbor Biosciences (myBaits Custom Capture Panel, Arbor Biosciences). DNA libraries from 24 samples were multiplexed in one capture reaction. Equal amounts of DNA libraries for each sample were used. We had also included probes to cover human autosomal regions for reference. Since EBV DNA is the minority in the plasma DNA pool, an ˜100× more excess of EBV probes relative to the autosomal DNA probes were used in each capture reaction. After the capture reaction, the captured DNA libraries were re-amplified with 14 cycles of PCR.

In some embodiments, targeted capture can be performed using capture probes designed to bind to any portion of the EBV genome. In some embodiments, capture probes can be biotinylated, and magnetic beads (e.g., streptavidin coated beads) are used to pull down or enrich the capture probes hybridized to a nucleic acid target (e.g., an EBV genome fragment) after library preparation. In some embodiments, the panel of capture probes used can also target a portion of the human genome. For example, capture probes may be designed to hybridize to at least a portion of one or more chromosomes (e.g., either copy of chromosomes 1, 8, and/or 13). In some embodiments, at least about 1 mb, at least 5 mb, at least 10 mb, at least 20 mb, at least 30 mb, at least 40 mb, at least 50 mb, at least 60 mb, at least 70 mb, at least 80 mb, at least 90 mb, or at least 100 mb of the human genome is targeted using capture probes in the panel.

To analyze the cell-free human papilloma virus (HPV) DNA in plasma, targeted sequencing (e.g., specifically designed capture probes, amplification primers) can be used. For example, capture probes can cover the whole HPV genome, the whole hepatitis B virus (HBV) genome, the whole EBV genome and multiple genomic regions in the human genome (for example but not limited to, including regions on chr1, chr2, chr3, chr5, chr8, chr15, chr22). For each plasma sample analyzed, DNA was extracted from 1-4 mL plasma using the QIAamp Circulating Nucleic Acid kit. For each case, all extracted DNA was used for the preparation of the sequencing library using the TruSeq Nano library preparation kit. Eight cycles of PCR amplification was performed on the sequencing library using the Illumina TruSeq Nano PCR amplification kit. The amplification products were captured using the myBaits Custom Capture Panel (Arbor Biosciences) using the custom-designed probes covering the viral and human genomic regions stated above. After target capturing, 14 cycles of PCR amplification were performed and the products were sequenced using the Illumina NextSeq platform. For each sequencing run, 24 samples with unique sample barcodes were sequenced using the paired-end mode. Each DNA fragments would be sequenced 75 nucleotides from each of the two ends. After sequencing, the sequenced reads would be mapped to an artificially combined reference sequence which consists of the whole human genome (hg19), the whole HPV genome, the whole HBV genome and the whole EBV genomes. Sequenced reads mapping to unique position in the combined genomic sequence would be used for downstream analysis.

For example, capture probes may be designed to cover the whole EBV genome, the whole hepatitis B virus (HBV) genome, the whole human papillomavirus (HPV) genome and/or multiple genomic regions in the human genome (for example but not limited to including regions on chr1, chr2, chr3, chr5, chr8, chr15 and chr22). To efficiently capture viral DNA fragments from plasma, more probes hybridizing to viral genomes than human autosomal regions of interest may be used. In one embodiment, for whole viral genomes, on average 100 hybridizing probes covering each region with ˜200 bp in size (e.g., 100× tiling capturing probes). For the regions of interest of human genome, we designed on average 2 hybridizing probes covering each region with ˜200 bp in size (e.g., 2× tiling capturing probes).

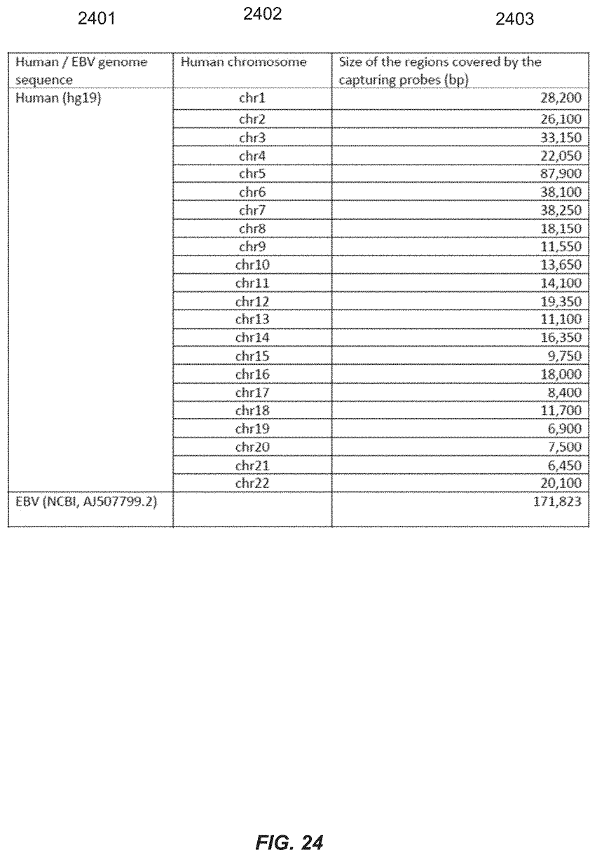

FIG. 24 shows an example of a design of capture probes for targeted capture sequencing of subjects, according to embodiments of the present disclosure. FIG. 24 provides information about capture probes, e.g., size of capturing regions and the amount of tiling covered by the probes. The capture probes can be various lengths and overlap with each other. Such capture probes can use the myBaits Custom Capture Panel (Arbor Biosciences). Other embodiments may not use such capture probes.

Referring to FIG. 24, target-capture sequencing for plasma EBV DNA was performed (Lam et al. Proc Natl Acad Sci U S A. 2018; 115:E5115-E5124) with modification in the design of the capture probes. The capture probes covered the whole EBV genome (AJ507799.2), in which the total size of the regions covered by the capturing probes is 171 kilobase. In addition, 467 kilobase of the human genome (hg19) was also captured for control.

Column 2401 identifies the type of sequence, i.e., autosomes of the human or viral targets. Column 2402 identifies the particular sequence (e.g., of a chromosome or of a particular viral genome). Column 2403 provides the total length in base pairs (bp) that the capture probes cover. The capture probes may not cover the entire sequence (e.g. as shown for the autosomes), but may cover the entire sequence, e.g., for a viral genome. For the autosomes, the capture probes provide 5× tiling on average. For the viral targets, the capture probes provide 200× tiling on average. Thus, the number of probes for the viral is a higher percentage/proportion per unit length than the autosomes. Such a higher level of concentration of capture probes for the viral targets can help maximize the chance of capturing the viral DNA.

### C. Example Results Using Various Target Capture Options

Various target capture options can be considered when enriching a biological sample for viral DNA. For example, a ratio between a viral genome and target autosomal regions can be configured to maximize detection of viral DNA from biological samples. An increased amount of plasma EBV DNA can be detected from the biological sample via targeted enrichment techniques, when capture probes are designed to target genomic regions that include a high ratio between target EBV genome and target autosomal regions. Optimizing enrichment techniques using different target capture options can thus facilitate a more accurate diagnosis of disease relapse in post-treatment samples.

DNA from plasma samples are subject to molecular analysis using next-generation sequencing (e.g., Illumina NextSeq platform). Different approaches can be used for targeted enrichment of DNA molecules that are of analytical interest, e.g., EBV DNA molecules. As described above, capture probes can be used for targeted enrichment of DNA molecules in a sample. Additionally or alternatively, amplicon sequencing (Xu et al. BMC Genomics 2017; 18:5. doi: 10.1186/s12864-016-3425-4) and CRISPR-Cas9 enrichment (Hafford-Tear et al. Genetics in Medicine 2019; 21:2092-2102) can be used to configure the ratio between the viral genome and target autosomal regions.

In target capture sequencing, capture probes are designed to cover the entire viral genome (e.g., EBV genome). Capture probes can also be included to target pre-defined genomic regions in the human genome. A ratio of the sizes of the target EBV genome and target autosomal regions can be configured to affect the degree of enrichment of EBV DNA molecules.

As an illustrative example, proportions of plasma EBV DNA obtained from target capture sequencing were determined using different capture probe designs (high and low EBV to target autosomal regions ratio). In addition, these designs were compared against proportions of plasma EBV DNA that were determined without using any target capture. Across three types of analyses, plasma samples with comparable concentrations of EBV DNA measured by quantitative PCR were used.

An illustrative example of a comparative analysis between various capture probe designs is presented as follows. With respect to the capture probes with “high” virus-to-human region size ratio, proportions of EBV DNA from 4 NPC plasma samples were determined using target capture sequencing with a capture probe design with an EBV to target autosomal region ratio of 0.37 (172 kb: 467 kb) (i.e. design described in Table 1). With respect to capture probes with “low” virus-to-human region size ratio, proportions of EBV DNA from another set of 4 NPC samples were determined using target capture sequencing with a capture probe design with an EBV to target autosomal region ratio of 0.0002 (172kb:70Mb). The NPC samples for of the above analyses had comparable plasma EBV DNA concentrations measured by quantitative PCR. For comparison data, proportion of EBV DNA from samples were also determined using sequencing without capture.

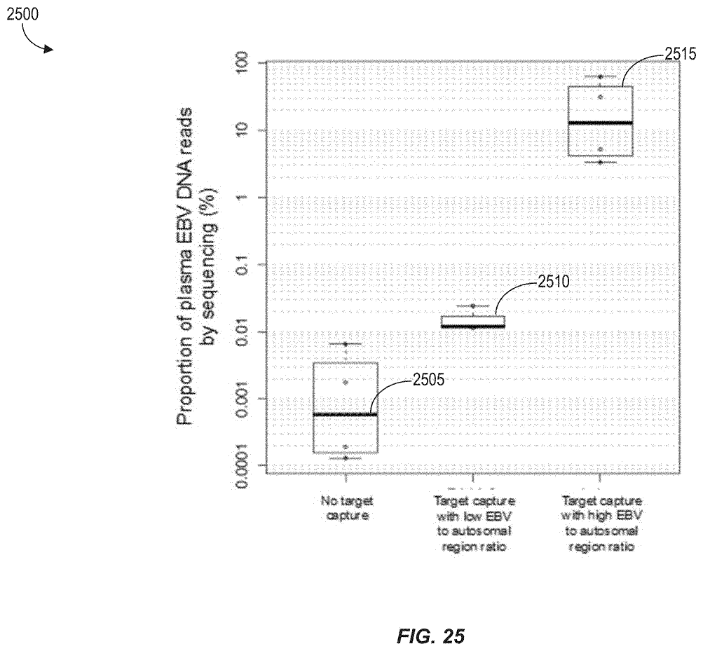

FIG. 25 shows a set of bar plots 2500 identifying plasma EBV DNA fractional concentration by sequencing of NPC samples that were analyzed by sequencing without target capture 2505, target capture sequencing using a probe design with low EBV to target autosomal region ratio 2510, and target capture sequencing using a design with high EBV to target autosomal region ratio 2515.

As shown in the bar plots 2510 and 2515, the proportions of plasma EBV DNA reads over the total number of sequenced reads were significantly higher when using target capture sequencing (post-hoc Dunn's test, p=0.005). Thus, enrichment of EBV DNA molecules through target capture sequencing can be effectively used for detecting viral DNA from post-treatment samples (for example). In contrast, as shown in the bar plot 2505, sequencing without target capture may yield low number of plasma EBV DNA reads and may hinder the subsequent downstream analysis (e.g., predicting disease relapse). For example, in sample TBR2892, sequencing without target capture may be less accurate for subsequent downstream analysis (e.g., the combined analysis as described in Section V). This is because, if sequencing without target capture is used, there would be no EBV DNA reads detected within the size range of 80 and 110 bp (out of 56 EBV DNA reads in total).

In addition, as shown in FIG. 25, the proportions of plasma EBV DNA reads over the total number of sequenced reads measured using the capture probe design with low EBV to target autosomal region ratio 2510 were substantially lower than those measured using the capture probe design with high EBV to target autosomal region ratio 2515.

### IX. TREATMENT BASED ON DISEASE-RELAPSE PREDICTION

### A. Treatment Selection

Embodiments of the present disclosure can accurately predict disease relapse, thereby facilitating early intervention and selection of appropriate treatments to improve disease outcome and overall survival rates of subjects. For example, an intensified chemotherapy can be selected for subjects, in the event their corresponding samples are predictive of disease relapse. In another example, a biological sample of a subject who had completed an initial treatment can be sequenced to identify viral DNA that is predictive of disease relapse. In such example, alternative treatment regimen (e.g., a higher dose) and/or a different treatment can be selected for the subject, as the subject's cancer may have been resistant to the initial treatment.

The embodiments may also include treating the subject in response to determining a classification of relapse of the pathology. For example, if the prediction corresponds to a loco-regional failure, surgery can be selected as a possible treatment. In another example, if the prediction corresponds to a distant metastasis, chemotherapy can be additionally selected as a possible treatment. In some embodiments, the treatment includes surgery, radiation therapy, chemotherapy, immunotherapy, targeted therapy, hormone therapy, stem cell transplant, or precision medicine. Based on the determined classification of relapse, a treatment plan can be developed to decrease the risk of harm to the subject and increase overall survival rate. Embodiments may further include treating the subject according to the treatment plan.

### B. Types of Treatments

Embodiments may further include treating the pathology in the patient after determining a classification for the subject. Treatment can be provided according to a determined level of pathology, the fractional concentration of clinically-relevant DNA, or a tissue of origin. For example, an identified mutation can be targeted with a particular drug or chemotherapy. The tissue of origin can be used to guide a surgery or any other form of treatment. And, the level of the pathology can be used to determine how aggressive to be with any type of treatment, which may also be determined based on the level of pathology. A pathology (e.g., cancer) may be treated by chemotherapy, drugs, diet, therapy, and/or surgery. In some embodiments, the more the value of a parameter (e.g., amount or size) exceeds the reference value, the more aggressive the treatment may be.

Treatment may include resection. For bladder cancer, treatments may include transurethral bladder tumor resection (TURBT). This procedure is used for diagnosis, staging and treatment. During TURBT, a surgeon inserts a cystoscope through the urethra into the bladder. The tumor is then removed using a tool with a small wire loop, a laser, or high-energy electricity. For patients with non-muscle invasive bladder cancer (NMIBC), TURBT may be used for treating or eliminating the cancer. Another treatment may include radical cystectomy and lymph node dissection. Radical cystectomy is the removal of the whole bladder and possibly surrounding tissues and organs. Treatment may also include urinary diversion. Urinary diversion is when a physician creates a new path for urine to pass out of the body when the bladder is removed as part of treatment.

Treatment may include chemotherapy, which is the use of drugs to destroy cancer cells, usually by keeping the cancer cells from growing and dividing. The drugs may involve, for example but are not limited to, mitomycin-C (available as a generic drug), gemcitabine (Gemzar), and thiotepa (Tepadina) for intravesical chemotherapy. The systemic chemotherapy may involve, for example but not limited to, cisplatin gemcitabine, methotrexate (Rheumatrex, Trexall), vinblastine (Velban), doxorubicin, and cisplatin.

In some embodiments, treatment may include immunotherapy. Immunotherapy may include immune checkpoint inhibitors that block a protein called PD-1. Inhibitors may include but are not limited to atezolizumab (Tecentriq), nivolumab (Opdivo), avelumab (Bavencio), durvalumab (Imfinzi), and pembrolizumab (Keytruda).

Treatment embodiments may also include targeted therapy. Targeted therapy is a treatment that targets the cancer's specific genes and/or proteins that contributes to cancer growth and survival. For example, erdafitinib is a drug given orally that is approved to treat people with locally advanced or metastatic urothelial carcinoma with FGFR3 or FGFR2 genetic mutations that has continued to grow or spread of cancer cells.

Some treatments may include radiation therapy. Radiation therapy is the use of high-energy x-rays or other particles to destroy cancer cells. In addition to each individual treatment, combinations of these treatments described herein may be used. In some embodiments, when the value of the parameter exceeds a threshold value, which itself exceeds a reference value, a combination of the treatments may be used. Information on treatments in the references are incorporated herein by reference.

### X. EXAMPLE SYSTEMS

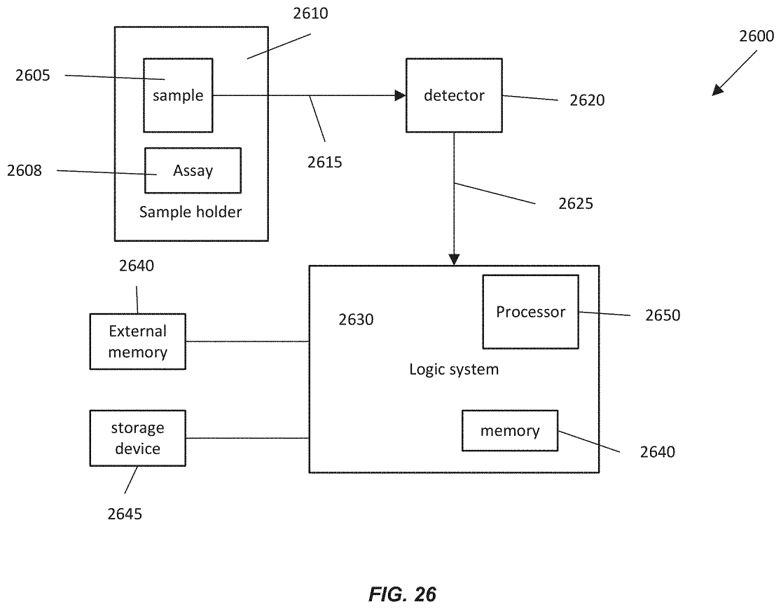

FIG. 26 illustrates a measurement system 2600 according to an embodiment of the present disclosure. The system as shown includes a sample 2605, such as cell-free DNA molecules within an assay device 2610, where an assay 2608 can be performed on sample 2605. For example, sample 2605 can be contacted with reagents of assay 2608 to provide a signal of a physical characteristic 2615. An example of an assay device can be a flow cell that includes probes and/or primers of an assay or a tube through which a droplet moves (with the droplet including the assay). Physical characteristic 2615 (e.g., a fluorescence intensity, a voltage, or a current), from the sample is detected by detector 2620. Detector 2620 can take a measurement at intervals (e.g., periodic intervals) to obtain data points that make up a data signal. In one embodiment, an analog-to-digital converter converts an analog signal from the detector into digital form at a plurality of times. Assay device 2610 and detector 2620 can form an assay system, e.g., a sequencing system that performs sequencing according to embodiments described herein. A data signal 2625 is sent from detector 2620 to logic system 2630. As an example, data signal 2625 can be used to determine sequences and/or locations in a reference genome of DNA molecules. Data signal 2625 can include various measurements made at a same time, e.g., different colors of fluorescent dyes or different electrical signals for different molecule of sample 2605, and thus data signal 2625 can correspond to multiple signals. Data signal 2625 may be stored in a local memory 2635, an external memory 2640, or a storage device 2645.

Logic system 2630 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2630 and the other components may be part of a stand-alone or network connected computer system, or they may be directly attached to or incorporated in a device (e.g., a sequencing device) that includes detector 2620 and/or assay device 2610. Logic system 2630 may also include software that executes in a processor 2650. Logic system 2630 may include a computer readable medium storing instructions for controlling measurement system 2600 to perform any of the methods described herein. For example, logic system 2630 can provide commands to a system that includes assay device 2610 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

System 2600 may also include a treatment device 2660, which can provide a treatment to the subject. Treatment device 2660 can determine a treatment and/or be used to perform a treatment. Examples of such treatment can include surgery, radiation therapy, chemotherapy, immunotherapy, targeted therapy, hormone therapy, and stem cell transplant. Logic system 2630 may be connected to treatment device 2660, e.g., to provide results of a method described herein. The treatment device may receive inputs from other devices, such as an imaging device and user inputs (e.g., to control the treatment, such as controls over a robotic system).

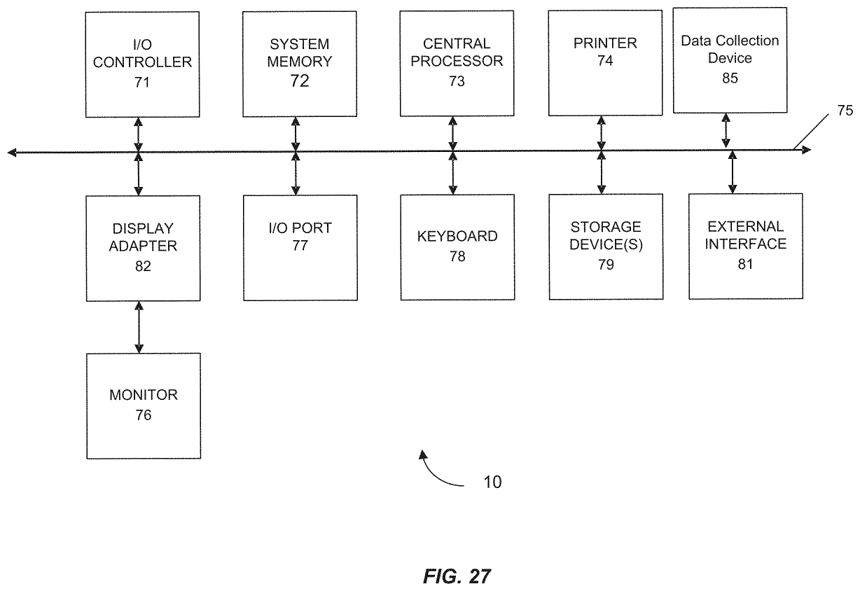

Any of the computer systems mentioned herein may utilize any suitable number of subsystems. Examples of such subsystems are shown in FIG. 27 in computer apparatus 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 27 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of connections known in the art such as input/output (I/O) port 77 (e.g., USB, FireWire®). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present invention using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or

Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

The section headings used herein are for organizational purposes only and are not to be construed as limiting the subject matter described.

It is to be understood that the methods described herein are not limited to the particular methodology, protocols, subjects, and sequencing techniques described herein and as such may vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention.

Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. The time constraint may be 1 minute, 1 hour, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”.

Whenever a range is given in the specification, for example, a temperature range, a time range, or a composition range, all intermediate ranges and subranges, as well as all individual values included in the ranges given are intended to be included in the disclosure.

As used herein, “consisting essentially of” does not exclude materials or steps that do not materially affect the basic and novel characteristics of the claim.

The claims may be drafted to exclude any element which may be optional. As such, this statement is intended to serve as antecedent basis for use of such exclusive terminology as “solely”, “only” and the like in connection with the recitation of claim elements, or the use of a “negative” limitation.

All patents, patent applications, publications, and descriptions mentioned herein are incorporated by reference in their entirety for all purposes. None is admitted to be prior art. Where a conflict exists between the instant application and a reference provided herein, the instant application shall dominate.

What is claimed is:

1. A method of analyzing a biological sample from a subject previously treated for a pathology and is currently asymptomatic for the pathology, the method comprising:
sequencing a first plurality of cell-free nucleic acid molecules from the mixture of nucleic acid molecules of the biological sample to obtain first sequence reads, wherein the biological sample includes a mixture of nucleic acid molecules from the subject and nucleic acid molecules from a virus;
attempting to align the first sequence reads to a reference genome, the reference genome corresponding to the virus;
determining an amount of the first sequence reads that align to the reference genome;
comparing the amount to a first cutoff; and
determining a classification for a relapse of the pathology based on the comparing of the amount to the first cutoff.

2. The method of claim 1, further comprising:
for each of a second plurality of cell-free nucleic acid molecules from the mixture of nucleic acid molecules of the biological sample of the subject:
measuring a size of the cell-free nucleic acid molecule; and
determining whether the cell-free nucleic acid molecule is from the reference genome;
determining a statistical value derived from the measured sizes of the second plurality of cell-free nucleic acid molecules that are from the reference genome; and
comparing the statistical value to a second cutoff, wherein determining the classification for the relapse of the pathology is further based on the comparing of the amount to the first cutoff and the comparing of the statistical value to the second cutoff

3. The method of claim 2, wherein measuring the size of the cell-free nucleic acid molecule includes sequencing the second plurality of cell-free nucleic acid molecules from the mixture of nucleic acid molecules of the biological sample to obtain second sequence reads, wherein the size of the cell-free nucleic acid molecule is measured using the second sequence reads.

4. The method of claim 2, wherein the first plurality of cell-free nucleic acid molecules is the second plurality of cell-free nucleic acid molecules.

5. The method of claim 2, wherein the statistical value includes a ratio of:
a first proportion of the second plurality of cell-free nucleic acid molecules that are from the reference genome of the virus with the size within a given range; and
a second proportion of the second plurality of cell-free nucleic acid molecules that are from a human reference genome with the size within the given range.

6. The method of claim 1, wherein the first cutoff and the second cutoff are determined from training samples having a known classification of the relapse.

7. The method of claim 1, wherein the pathology is a cancer.

8. The method of claim 7, wherein the cancer is selected from a group consisting of nasopharyngeal cancer, head and neck squamous cell carcinoma, cervical cancer, and hepatocellular carcinoma.

9. The method of claim 1, further comprising enriching the biological sample for nucleic acid molecules from the virus.

10. The method of claim 1, wherein the virus comprises EBV DNA, HPV DNA, HBV DNA, HCV nucleic acids, or fragments thereof.

11. The method of claim 1, wherein the subject is a pregnant woman.

12. The method of claim 1, further comprising:
responsive to determining the classification, initiating another treatment to the subject to prevent the relapse of the pathology.

13. The method of claim 1, wherein the classification comprises remission, relapse, loco-regional failure, or distant metastasis.

14. A method of analyzing a biological sample from a subject previously treated for a pathology and is currently asymptomatic for the pathology, the method comprising:
performing a first assay, wherein the first assay comprises analyzing a first plurality of cell-free nucleic acid molecules from the mixture of nucleic acid molecules of the biological sample of the subject, wherein the biological sample includes a mixture of nucleic acid molecules from the subject and nucleic acid molecules from a virus;
performing a second assay, wherein the second assay comprises:
for each of a second plurality of cell-free nucleic acid molecules from the mixture of nucleic acid molecules of the biological sample of the subject:
measuring a size of the cell-free nucleic acid molecule; and
determining whether the cell-free nucleic acid molecule is from a reference genome, the reference genome corresponding to the virus;
determining an amount of the first plurality of cell-free nucleic acid molecules that align to the reference genome;
determining a statistical value derived from the measured sizes of the second plurality of cell-free nucleic acid molecules that are from the reference genome;
comparing the amount to a first cutoff;
comparing the statistical value to a second cutoff; and
determining a classification for a relapse of the pathology based on the comparing of the amount to the first cutoff and the comparing of the statistical value to the second cutoff

15. The method of claim 14, wherein measuring the size of the cell-free nucleic acid molecule includes sequencing the second plurality of cell-free nucleic acid molecules from the mixture of nucleic acid molecules of the biological sample to obtain sequence reads, wherein the size of the cell-free nucleic acid molecule is measured using the sequence reads.

16. The method of claim 14, wherein the first assay includes real-time PCR, digital PCR, or sequencing.

17. The method of claim 14, wherein the first plurality of cell-free nucleic acid molecules is the second plurality of cell-free nucleic acid molecules.

18. The method of claim 14, wherein the statistical value includes a ratio of:
a first proportion of the second plurality of cell-free nucleic acid molecules that are from the reference genome of the virus with the size within a given range; and
a second proportion of the second plurality of cell-free nucleic acid molecules that are from a human reference genome with the size within the given range.

19. The method of claim 14, wherein the first cutoff and the second cutoff are determined from training samples having a known classification of the relapse.

20. The method of claim 14, wherein the pathology is a cancer.

21. The method of claim 20, wherein the cancer is selected from a group consisting of nasopharyngeal cancer, head and neck squamous cell carcinoma, cervical cancer, and hepatocellular carcinoma.

22. The method of claim 14, further comprising enriching the biological sample for nucleic acid molecules from the virus.

23. The method of claim 14, wherein the virus comprises EBV DNA, HPV DNA, HBV DNA, HCV nucleic acids, or fragments thereof.

24. The method of claim 14, wherein the subject is a pregnant woman.

25. The method of claim 14, further comprising:
responsive to determining the classification, initiating another treatment to the subject to prevent the relapse of the pathology.

26. The method of claim 14, wherein the classification comprises remission, relapse, loco-regional failure, or distant metastasis.
